SEQUENCE LISTING (26 sequences)

SEQ ID NO: 1 - DNA, 22 bp, Artificial Sequence
  Oligonucleotide Primer
tgcaatcaatcccagtattttg

SEQ ID NO: 2 - DNA, 22 bp, Artificial Sequence
  Oligonucleotide Primer
cttgtgcaagcataagtctcaa

SEQ ID NO: 3 - DNA, 27 bp, Artificial Sequence
  Oligonucleotide Primer
gaagttggtgggaaaatgagtccgtga

SEQ ID NO: 4 - DNA, 27 bp, Artificial Sequence
  Oligonucleotide Primer
gccatggctttgccgtagtagattctg

SEQ ID NO: 5 - DNA, 22 bp, Artificial Sequence
  Oligonucleotide Primer
acgggagatcaaaggctgctct

SEQ ID NO: 6 - DNA, 19 bp, Artificial Sequence
  Oligonucleotide Primer
gccaacaaggtgcttttgc

SEQ ID NO: 7 - DNA, 20 bp, Artificial Sequence
  Oligonucleotide Primer
ctcaccgtgacgttttaggg

SEQ ID NO: 8 - DNA, 21 bp, Artificial Sequence
  Oligonucleotide Primer
gtgaatgatatgacatcagac

SEQ ID NO: 9 - DNA, 27 bp, Artificial Sequence
  Oligonucleotide Primer
cgacgaacatgctcccctgatgttttt

SEQ ID NO: 10 - DNA, 27 bp, Artificial Sequence
  Oligonucleotide Primer
aaactcatagatttctgcaggacagcc

SEQ ID NO: 11 - DNA, 21 bp, Artificial Sequence
  Oligonucleotide Primer
agctgcatctctttctctcca

SEQ ID NO: 12 - DNA, 22 bp, Artificial Sequence
  Oligonucleotide Primer
tatcctgaaggccaaaatagga

SEQ ID NO: 13 - DNA, 21 bp, Artificial Sequence
  Oligonucleotide Primer
acgaccagaacatccagaaga

SEQ ID NO: 14 - DNA, 20 bp, Artificial Sequence
  Oligonucleotide Primer
gcagagaaagaaatccccgc

SEQ ID NO: 15 - DNA, 22 bp, Artificial Sequence
  Oligonucleotide Primer
agggggacttgctttgccattt

SEQ ID NO: 16 - DNA, 24 bp, Artificial Sequence
  Oligonucleotide Primer
aacaccccttctttcctcctccat

SEQ ID NO: 17 - DNA, 20 bp, Artificial Sequence
  Oligonucleotide Primer
ctgctccttcctgccttcta

SEQ ID NO: 18 - DNA, 20 bp, Artificial Sequence
  Oligonucleotide Primer
ctgagccaggtttcatgtga

SEQ ID NO: 19 - DNA, 21 bp, Artificial Sequence
  Oligonucleotide Primer
actcctacgggaggcagcagt

SEQ ID NO: 20 - DNA, 18 bp, Artificial Sequence
  Oligonucleotide Primer
attaccgcggctgctggc

SEQ ID NO: 21 - DNA, 18 bp, Artificial Sequence
  Oligonucleotide Primer
actcctacgggaggcagc

SEQ ID NO: 22 - DNA, 23 bp, Artificial Sequence
  Oligonucleotide Primer
gcttcttagtcaggtaccgtcat

SEQ ID NO: 23 - DNA, 19 bp, Artificial Sequence
  Oligonucleotide Primer
ggttctgagaggaggtccc

SEQ ID NO: 24 - DNA, 18 bp, Artificial Sequence
  Oligonucleotide Primer
gctggctcccgtaggagt

SEQ ID NO: 25 - DNA, 19 bp, Artificial Sequence
  Oligonucleotide Primer
ggttctgagaggaggtccc

SEQ ID NO: 26 - DNA, 18 bp, Artificial Sequence
  Oligonucleotide Primer
gctggctcccgtaggagt

(19) United States
(12) Patent Application Publication
(10) Pub. No.: US 2019/0142868 A1
(43) Pub. Date: May 16, 2019

(54) METHODS OF TREATING DISEASES ASSOCIATED WITH ILC3 CELLS

(71) Applicant: Instituto de Medicina Moleccular, Lisboa (PT)

(72) Inventors: José Henrique Veiga Fernandes, Oeiras (PT); Sales Ibiza Martinez, Lisboa (PT); Bethania García-Cassani, Lisboa (PT)

(73) Assignee: Instituto de Medicina Moleccular, Lisboa (PT)

(21) Appl. No.: 16/300,338

(22) PCT Filed: May 11, 2017
(86) PCT No.: PCT/IB2017/000901

§ 371 Date: Nov. 9, 2018

(30) Foreign Application Priority Data
May 13, 2016 (PT) 20161000032176

(51) Int. Cl. A61K 35/17; A61K 39/395; A61K 38/18; A61K 38/17; C12N 5/078; C12N 15/113

(52) U.S. Cl. CPC A61K 35/17; A61K 39/3955; A61K 38/185; A61K 38/179; C12N 5/0634; C12N 2310/122; C12N 15/1138; C12N 15/1136; C12N 2501/13; C12N 2501/998; C12N 2310/11; C12N 15/1137

(57) ABSTRACT

Provided herein are compositions including compounds and/or cells for treating a disease associated with Group 3 innate lymphoid cells (ILC3s), and methods of treatment.

### BACKGROUND

Group 3 innate lymphoid cells (ILC3) are major regulators of inflammation and infection at mucosal barriers¹. ILC3 development has been considered to be programmed¹. Nevertheless, how ILC3 perceive, integrate and respond to local environmental signals remains unclear.

### SUMMARY

As shown herein, ILC3 sense their environment and control gut defence as part of a novel glial-ILC3-epithelial cell unit orchestrated by neurotrophic factors. As further shown herein, enteric ILC3 express the neuroregulatory receptor rearranged during transfection (RET). ILC3-autonomous Ret ablation led to decreased innate interleukin-22 (IL-22), impaired epithelial reactivity, dysbiosis and increased susceptibility to bowel inflammation and infection. Neurotrophic factors directly controlled innate Il22, downstream of p38 MAPK/ERK-AKT cascade and STAT3 activation. Strikingly, ILC3 were adjacent to neurotrophic factor expressing glial cells that exhibited stellate-shaped projections into ILC3 aggregates. Glial cells sensed microenvironmental cues in a MYD88 dependent manner to control neurotrophic factors and innate IL-22. Accordingly, glial-intrinsic Myd88 deletion led to impaired ILC3-derived IL-22 and pronounced propensity to gut inflammation and infection. This work sheds light into a novel multi-tissue defence unit, revealing glial cells as central hubs of neuron and innate immune regulation via neurotrophic factor signals.

According to one aspect, methods for increasing production of interleukin-22 (IL-22) by Group 3 innate lymphoid cells (ILC3s) are provided. The methods include contacting ILC3s with an agonist of rearranged during transfection (RET) in an amount effective to increase production of IL-22 by the ILC3s.

In some embodiments, the agonist of RET includes (1) a combination of a soluble GDNF Family binding Receptor alpha (GFRα) and a GFRα ligand (GFL) or an analog or mimetic thereof; or (2) an antibody that specifically binds to RET and increases RET tyrosine kinase activity or an antigen-binding fragment thereof. In some embodiments, the combination of a soluble GDNF Family binding Receptor alpha (GFRα) and a GFRα ligand or an analog or mimetic thereof includes: (1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or (2) a combination of two or more of (a), (b), (c), (d), (e), (f) and (g).

In some embodiments, the contacting is in vitro. In some embodiments, the contacting is in vivo.

In some embodiments, the agonist is administered to a subject. In some embodiments, the subject is a human. In some embodiments, the subject is not otherwise in need of treatment with the agonist.

According to another aspect, methods for treating a disease associated with Group 3 innate lymphoid cells (ILC3s) are provided. The methods include administering to a subject in need of such treatment an agonist of rearranged during transfection (RET) in an amount effective to treat the disease.

In some embodiments, the agonist of RET includes (1) a combination of a soluble GDNF Family binding Receptor alpha (GFRα) and a GFRα ligand or an analog or mimetic thereof; or (2) an antibody that specifically binds to RET and increases RET tyrosine kinase activity or an antigen-binding fragment thereof. In some embodiments, the combination of a soluble GDNF Family binding Receptor alpha (GFRα) and a GFRα ligand or an analog or mimetic thereof includes: (1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or (2) a combination of two or more of (a), (b), (c), (d), (e), (f) and (g).

In some embodiments, the subject is a human.

In some embodiments, the disease is infection, inflammation, neoplasia, or altered gut physiology.

In some embodiments, the subject is not otherwise in need of treatment with the agonist of RET.

In some embodiments, the agonist of RET is administered intravenously, orally, nasally, rectally or through skin absorption.

According to another aspect, agonists of rearranged during transfection (RET) are provided for use in treating a disease associated with Group 3 innate lymphoid cells (ILC3s), including administering to a subject in need of such treatment the agonist of RET in an amount effective to treat the disease.

In some embodiments, the agonist of RET includes (1) a combination of a soluble GDNF Family binding Receptor alpha (GFRα) and a GFRα ligand or an analog or mimetic thereof; or (2) an antibody that specifically binds to RET and increases RET tyrosine kinase activity or an antigen-binding fragment thereof. In some embodiments, the combination of a soluble GDNF Family binding Receptor alpha (GFRα) and a GFRα ligand or an analog or mimetic thereof includes: (1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or (2) a combination of two or more of (a), (b), (c), (d), (e), (f) and (g).

In some embodiments, the subject is a human.

In some embodiments, the disease is infection, inflammation, neoplasia, or altered gut physiology.

In some embodiments, the subject is not otherwise in need of treatment with the agonist of RET.

In some embodiments, the agonist of RET is administered intravenously, orally, nasally, rectally or through skin absorption.

According to another aspect, methods for treating a disease associated with Group 3 innate lymphoid cells (ILC3s) are provided. The methods include administering to a subject in need of such treatment a composition including ILC3s in an amount effective to treat the disease.

In some embodiments, the composition further includes an agonist of rearranged during transfection (RET). In some embodiments, the agonist of RET includes (1) a combination of a soluble GDNF Family binding Receptor alpha (GFRα) and a GFRα ligand or an analog or mimetic thereof; or (2) an antibody that specifically binds to RET and increases RET tyrosine kinase activity or an antigen-binding fragment thereof. In some embodiments, the combination of a soluble GDNF Family binding Receptor alpha (GFRα) and a GFRα ligand or an analog or mimetic thereof includes: (1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or (2) a combination of two or more of (a), (b), (c), (d), (e), (f) and (g).

In some embodiments, the subject is a human.

In some embodiments, the disease is infection, inflammation, neoplasia, or altered gut physiology.

In some embodiments, the subject is not otherwise in need of treatment with the ILC3s or the agonist of RET.

In some embodiments, the ILC3s or the agonist of RET is administered intravenously, orally, nasally, rectally or through skin absorption.

According to another aspect, compositiond including activated Group 3 innate lymphoid cells (ILC3s) are provided for use in treating a disease associated with ILC3s including administering to a subject in need of such treatment the composition including ILC3s in an amount effective to treat the disease.

In some embodiments, the composition further includes an agonist of rearranged during transfection (RET). In some embodiments, the agonist of RET includes (1) a combination of a soluble GDNF Family binding Receptor alpha (GFRα) and a GFRα ligand or an analog or mimetic thereof; or (2) an antibody that specifically binds to RET and increases RET tyrosine kinase activity or an antigen-binding fragment thereof. In some embodiments, the combination of a soluble GDNF Family binding Receptor alpha (GFRα) and a GFRα ligand or an analog or mimetic thereof includes: (1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or (2) a combination of two or more of (a), (b), (c), (d), (e), (f) and (g).

In some embodiments, the subject is a human.

In some embodiments, the disease is infection, inflammation, neoplasia, or altered gut physiology.

In some embodiments, the subject is not otherwise in need of treatment with the ILC3s or the agonist of RET.

In some embodiments, the ILC3s or the ILC3s and the agonist of RET is administered intravenously, orally, nasally, rectally or through skin absorption.

According to another aspect, methods for decreasing production of interleukin-22 (IL-22) by Group 3 innate lymphoid cells (ILC3s) are provided. The methods include contacting ILC3s with an antagonist of rearranged during transfection (RET) in an amount effective to decrease production of IL-22 by the ILC3s.

In some embodiments, the antagonist of RET is (1) an antibody that specifically binds and inhibits: (a) RET tyrosine kinase activity, (b) a GDNF Family binding Receptor alpha (GFRα), or (c) a GFRα ligand, or an antigen-binding fragment thereof; (2) an inhibitory nucleic acid molecule that reduces expression, transcription or translation of RET, a GFRα, or a GFRα ligand; or (3) a RET tyrosine kinase inhibitor, optionally AST 487, motesanib, cabozantinib, vandetanib, ponatinib, sunitinib, sorafenib, or alectinib. In some embodiments, the GFRα is GFRα1, GFRα2, GFRα3, or GFRα4; or wherein the GFRα ligand is glial cell line-derived neurotrophic factor (GDNF), neurturin (NTRN), artemin (ARTN), or persephin (PSPN). In some embodiments, the inhibitory nucleic acid molecule is a sRNA, shRNA, or antisense nucleic acid molecule.

In some embodiments, the contacting is in vitro. In some embodiments, the contacting is in vivo.

In some embodiments, the antagonist of RET is administered to a subject. In some embodiments, the subject is a human. In some embodiments, the subject is not otherwise in need of treatment with the antagonist of RET.

According to another aspect, methods for treating a disease associated with Group 3 innate lymphoid cells (ILC3s) are provided. The methods include administering to a subject in need of such treatment an antagonist of rearranged during transfection (RET) in an amount effective to treat the disease.

In some embodiments, the antagonist of RET is (1) an antibody that specifically binds and inhibits: (a) RET tyrosine kinase activity, (b) a GDNF Family binding Receptor alpha (GFRα), or (c) a GFRα ligand, or an antigen-binding fragment thereof; (2) an inhibitory nucleic acid molecule that reduces expression, transcription or translation of RET, a GFRα, or a GFRα ligand; or (3) a RET tyrosine kinase inhibitor, optionally AST 487, motesanib, cabozantinib, vandetanib, ponatinib, sunitinib, sorafenib, or alectinib. In some embodiments, the GFRα is GFRα1, GFRα2, GFRα3, or GFRα4; or wherein the GFRα ligand is glial cell line-derived neurotrophic factor (GDNF), neurturin (NTRN), artemin (ARTN), or persephin (PSPN). In some embodiments, the inhibitory nucleic acid molecule is a sRNA, shRNA, or antisense nucleic acid molecule.

In some embodiments, the subject is a human.

In some embodiments, the subject is not otherwise in need of treatment with the antagonist of RET.

In some embodiments, the disease is epithelial intestinal cancer.

In some embodiments, the antagonist of RET is administered intravenously, orally, nasally, rectally or through skin absorption.

According to another aspect, antagonists of rearranged during transfection (RET) are provided for use in treating a disease associated with Group 3 innate lymphoid cells (ILC3) including administering to a subject in need of such treatment the antagonist of RET in an amount effective to treat the disease.

In some embodiments, the antagonist of RET is (1) an antibody that specifically binds and inhibits: (a) RET tyrosine kinase activity, (b) a GDNF Family binding Receptor alpha (GFRα), or (c) a GFRα ligand, or an antigen-binding fragment thereof; (2) an inhibitory nucleic acid molecule that reduces expression, transcription or translation of RET, a GFRα, or a GFRα ligand; or (3) a RET tyrosine kinase inhibitor, optionally AST 487, motesanib, cabozantinib, vandetanib, ponatinib, sunitinib, sorafenib, or alectinib. In some embodiments, the GFRα is GFRα1, GFRα2, GFRα3, or GFRα4; or wherein the GFRα ligand is glial cell line-derived neurotrophic factor (GDNF), neurturin (NTRN), artemin (ARTN), or persephin (PSPN). In some embodiments, the inhibitory nucleic acid molecule is a sRNA, shRNA, or antisense nucleic acid molecule.

In some embodiments, the subject is a human.

In some embodiments, the subject is not otherwise in need of treatment with the antagonist of RET.

In some embodiments, the disease is epithelial intestinal cancer.

In some embodiments, the antagonist of RET is administered intravenously, orally, nasally, rectally or through skin absorption.

The invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1a-1h. The neurotrophic factor receptor RET drives enteric ILC3-derived IL-22. FIG. 1a, LTi, NCR⁻ and NCR⁺ILC3 subsets, T cells (T), B cells (B), Dendritic cells (Dc), Macrophages (Mø), enteric Neurons (N) and mucosal Glial cells (G). FIG. 1b, Ret^{GFP} ILC3. FIG. 1c, Left: Ret^{GFP} gut. White: GFP. Right: ILC3 aggregates. FIG. 1d, Cryptopatches (CP), immature (iILF) and mature (mILF) isolated lymphoid follicles. Green: RET/GFP; Blue: RORγt; Red: B220. FIG. 1e, Ret^{GFP} chimeras. n=15. FIGS. 1f,1g, Ret^{GFP} chimeras. Ret^{WT/GFP} n=25; Ret^{GFP/GFP} n=22. FIG. 1h, Ret^{MEN2B} mice. n=7. Scale bars: 1 mm (c left, e); 50 μm (c right); 30 μm (d). Data are representative of 4 independent experiments. Error bars show s.e.m.*P<0.05; **P<0.01; ns not significant.

FIGS. 2a-2n. ILC3-intrinsic RET signals regulate gut defence. FIG. 2a, ILC3-derived cytokines. n=11. FIG. 2b, Ret^{Δ} and Ret^{MEN2B} mice compared to their WT littermate controls. n=7. FIGS. 2c-2f, DSS treatment. Ret^{fl} n=8; Ret^{Δ} n=8. c, Histopathology. FIG. 2d, Inflammation score and colon length. FIG. 2e, Innate IL-22. FIG. 2f, Bacterial translocation. FIGS. 2g-2j, DSS treatment. Ret^{WT} n=8; Ret^{MEN2B} n=8. FIG. 2g, Histopathology. FIG. 2h, Inflammation score and colon length. FIG. 2i, Innate IL-22. FIG. 2j, Bacterial translocation. FIGS. 2k-2n, C. rodentium infection. Rag1^{-/-}.Ret^{fl} n=15; Rag1^{-/-}.Ret^{Δ} n=17. FIG. 2k, Histopathology. FIG. 2l, Inflammation score and colon length. FIG. 2m, Innate IL-22. FIG. 2n, Infection burden. Scale bars: 200 μm. Data are representative of 4 independent experiments. Error bars show s.e.m.*P<0.05; **P<0.01; ns not significant.

FIGS. 3a-3j. ILC3-autonomous RET signals directly control Il22 downstream of pSTAT3. FIGS. 3a,3b, Epithelial/ILC3 organoids. n=9. FIG. 3c, Ret^{Δ} ILC3 compared to their WT controls. n=4. FIG. 3d, ILC3 activation by GFL. n=4. FIG. 3e, Ret^{Δ} ILC3. pERK n=8; pAKT n=12; phosphorylated p38/MAP kinase n=6; pSTAT3 n=14. FIG. 3f, ILC3 activation by GFL. pERK n=10; pAKT n=16; phosphorylated p38/MAP kinase n=3; pSTAT3 n=15. FIG. 3g, pSTAT3 in ILC3 cultured with medium (n=7), GFL (n=1) or GFL and inhibitors for: p38 MAPK/ERK-AKT (LY) (n=7); ERK (PD) (n=7); AKT (VIII) (n=8); and p38 MAPK (SB) (n=6). FIG. 3h, Il22 in ILC3 cultured with GFL (n=17) or GFL and the inhibitors LY (n=18); PD (n=16); VIII (n=15); SB (n=15); and the STAT3 inhibitor (S3I) (n=8). FIG. 3i, Il22 locus. FIG. 3j. ChIP analysis of ILC3 stimulated with GFL. n=10. Data are representative of 3 independent experiments. Error bars show s.e.m.*P<0.05; **P<0.01; ns not significant.

FIGS. 4a-4m. Glial cells set GFL expression and innate IL-22, via MYD88-dependent sensing of the microenvironment. FIG. 4a, Weighted Unifrac PCoA analysis and genus-level comparisons from co-housed Ret^{fl} (white circles) and Ret^{Δ} (black circles) littermates. n=5. Genera from bottom to top: Purple: Unclassified S24-7; Red: Bacteroides; Blue: Unclassified Clostridiales; Green: Sutterella; Grey: Other. FIGS. 4b-4d, DSS treatment of colonised germ-free (GF) mice. n=5. FIG. 4b, Histopathology. FIG. 4c, Inflammation score. FIG. 4d, Innate IL-22. FIG. 4e, Innate IL-22 after antibiotic treatment. n=8. FIG. 4f, Ret^{GFP}.Gfap-Cre.Rosa26^{RFP} mice. Green: RET/GFP; Red: GFAP/RFP. FIGS. 4g,4h, Glial cell activation with TLR2, TLR4, IL-1β receptor and IL-33 receptor ligands. n=6. FIG. 4i, TLR ligands, IL-1β and IL-33 activation of co-cultured ILC3 with WT (white bars) or Myd88^{-/-} glial cells (black bars). n=6. FIGS. 4j-4m, DSS treatment of Gfap-Cre.Myd88^{Δ} mice. n=12. FIG. 4j, Histopathology. FIG. 4k, Inflammation score and colon length. FIG. 4l, Innate IL-22. FIG. 4m, Body weight. Scale bars: 200 μm (b, j); 10 am (f). Data are representative of 3-4 independent experiments. Error bars show s.e.m.*P<0.05; **P<0.01; ns not significant.

FIGS. 5a-5j. ILC3 selectively express the neurotrophic factor receptor RET. FIG. 5a, Expression of RET protein in gut CD45⁺Lin⁻Thy1.2^{hi}IL7Rα⁺RORγt⁺ILC3. FIG. 5b, Analysis of gut ILC3 from Ret^{GFP} mice. Embryonic day 14.5 (E14.5). FIGS. 5c,5d Analysis of enteric ILC3 subsets from Ret^{GFP} mice. FIG. 5e, Analysis of cytokine producing ILC3 from Ret^{GFP} mice. FIG. 5f, Pregnant Ret^{GFP} mice were provided with antibiotic cocktails that were maintained after birth until analysis at 6 weeks of age. Left: RET/GFP (white). Right: flow cytometry analysis of RET/GFP expression in ILC3. Thin line: Ab treated; Bold line: SPF. FIG. 5g, Ret expression in enteric ILC3 from Germ-Free (GF) mice and Specific Pathogen Free (SPF) controls. n=4. FIG. 5h, Analysis of lamina propria populations from Ret^{GFP} mice. FIG. 5i, Enteric ILC3 clusters. Green: RET/GFP; Blue: RORγt; Red: B220. Bottom: quantification analysis for RET/GFP and RORγt co-expression (79,97±4,72%). FIG. 5j, Rare RET expressing ILC3 in intestinal villi. Green: RET/GFP; Blue: RORγt; Red: CD3ε. Scale bars: 10 μm. Data are representative of 4 independent experiments. Error bars show s.e.m. ns not significant.

FIGS. 6a-6b. T cell-derived IL-22 and IL-17 in Ret^{GFP} chimeras and Ret^{MEN2B} mice. FIG. 6a, T cell derived IL-17 in Ret^{GFP} chimeras. Ret^{WT/GFP} n=25; Ret^{GFP/GFP} n=22. FIG. 6b, T cell derived IL-22 and IL17 in the intestine of Ret^{MEN2B} mice and their WT littermate controls. Ret^{WT} n=7; Ret^{MEN2B} n=7. Data are representative of 4 independent experiments. Error bars show s.e.m. ns not significant.

FIGS. 7a-7i. Enteric homeostasis in steady-state Ret^{Δ} mice. FIG. 7a, Rorgt-Cre mice were bread to Rosa26^{YFP}. Analysis of Rosa26/YFP expression in gut ILC3 from Rorgt-Cre.Rosa26^{YFP} mice. FIG. 7b, Number of Peyer's patches (PP). Ret^{fl} n=10; Ret^{Δ} n=10. FIG. 7c, T cell derived IL-22 in Ret^{Δ} mice and their WT littermate controls. Ret^{fl} n=11; Ret^{Δ} n=11. FIG. 7d, γδ T cell derived IL-22 in Ret^{Δ} mice and their WT littermate controls. Ret^{fl} n=4; Ret^{Δ} n=4. FIG. 7e, Intestinal villus and crypt morphology. Ret^{fl} n=6; Ret^{Δ} n=6. FIG. 7f, Epithelial cell proliferation. Ret^{fl} n=5; Ret^{Δ} n=5. FIG. 7g, Intestinal paracellular permeability measured by Dextran-Fitc in the plasma. Ret^{fl} n=5; Ret^{Δ} n=5. FIG. 7h, Tissue repair genes in Ret^{Δ} intestinal epithelium in comparison to their WT littermate controls. n=8. FIG. 7i, Reactivity genes in Ret^{MEN2B} mice treated with anti-IL-22 blocking antibodies in comparison to Ret^{MEN2B} intestinal epithelium. Ret^{MEN2B} n=4; Ret^{MEN2B}+anti-IL-22 n=4. Data are representative of 3 independent experiments. Error bars show s.e.m. ns not significant.

FIGS. 8a-8g. Enteric inflammation in mice with altered RET signals. Mice were treated with DSS in the drinking water. FIG. 8a, Weight loss of DSS treated Ret^{Δ} mice and their littermate controls. Ret n=8; Ret^{Δ} n=8. FIG. 8b, T cell derived IL-22 in Ret^{Δ} mice and their WT littermate controls after DSS treatment. Ret^{fl} n=8; Ret^{Δ} n=8. FIG. 8c, Weight loss of DSS treated Ret^{MEN2B} mice and their WT littermate controls. Ret^{WT} n=8; Ret^{MEN2B} n=8. FIG. 8d, T cell derived IL-22 in Ret^{MEN2B} mice and their WT littermate controls. Ret^{WT} n=8; Ret^{MEN2B} n=8. FIG. 8e, Intestinal villi and crypt morphology. Ret^{fl} n=6; Ret^{Δ} n=6. FIG. 8f, Epithelial reactivity gene expression in DSS treated Ret^{Δ} mice in comparison to their WT littermate controls. n=8. FIG. 8g, Tissue repair gene expression in DSS treated Ret^{Δ} mice in comparison to their WT littermate controls. n=4. Data are representative of 3-4 independent experiments. Error bars show s.e.m. ns not significant. Error bars show s.e.m.*P<0.05; **P<0.01; ns not significant.

FIGS. 9a-9k. Citrobacter rodentium infection in Ret^{Δ} mice. FIG. 9a, C. rodentium translocation to the liver of Rag1^{-/-}.Ret^{Δ} and their Rag1^{-/-}.Ret^{fl} littermate controls at day 6 post-infection. n=15. FIG. 9b, MacConkey plates of liver cell suspensions from Rag1^{-/-}.Ret^{Δ} and their Rag1−^{-/-}.Ret^{fl} littermate controls at day 6 after C. rodentium infection. FIG. 9c, Whole-body imaging of Rag1^{-/-}.Ret^{Δ} and their Rag1^{-/-}.Ret littermate controls at day 6 after luciferase-expressing C. rodentium infection. FIG. 9d, Epithelial reactivity gene expression in C. rodentium infected Rag1^{-/-}.Ret^{Δ} mice and their Rag1^{-/-}.Ret^{fl} littermate controls. Rag1^{-/-}.Ret^{fl} n=15; Rag1^{-/-}.Ret^{Δ} n=17. FIG. 9e, Weight loss in C. rodentium infected Rag1^{-/-}.Ret^{Δ} mice and their Rag1^{-/-}.Ret littermate controls. Rag1^{-/-}.Ret n=8; Rag1^{-/-}.Ret^{Δ} n=8. FIG. 9f, Survival curves in C. rodentium infected Rag1^{-/-}.Ret^{Δ} mice and their Rag1^{-/-}.Ret littermate controls. Rag1^{-/-}.Ret^{fl} n=8; Rag1^{-/-}.Ret^{Δ} n=8. FIG. 9g, C. rodentium translocation to the liver of Ret^{Δ} and their Ret^{fl} littermate controls at day 6 post-infection. n=6. FIG. 9h, MacConkey plates of liver cell suspensions from Ret^{Δ} and their Ret^{fl} littermate controls at day 6 after C. rodentium infection. FIG. 9i, Whole-body imaging of Ret^{Δ} and their Ret^{fl} littermate controls at day 6 after luciferase-expressing C. rodentium infection. FIG. 9j, C. rodentium infection burden. Ret^{fl} n=8; Ret^{Δ} n=8. FIG. 9k, Innate IL-22 in in C. rodentium infected Ret^{Δ} mice and their Ret^{fl} littermate controls. Ret^{fl} n=8; Ret^{Δ} n=8. Data are representative of 3-4 independent experiments. Error bars show s.e.m. ns not significant. Error bars show s.e.m.*P<0.05; **P<0.01; ns not significant.

FIGS. 10a-10f. Glial-derived neurotrophic factor family ligand (GFL) signals in ILC3. FIG. 10a, Multi-tissue intestinal organoid system. Scale bar: 20 μm. Black arrows: ILC3. FIG. 10b, Expression of ILC-related genes in ILC3 from Ret^{Δ} mice in comparison to their littermate controls. n=4. FIG. 10c, ILC3 activation with all GFL/GFRα pairs (GFL); single GDNF family ligand (GDNF, ARTN or NRTN); or single GFL/GFRα pairs (GDNF/GFRα1, ARTN/GFRα3 or NRTN/GFRα2) compared to vehicle BSA. n=5. FIG. 10d, ILC3 from Ret^{Δ} mice (open black) and their littermate controls (open dash). Isotype (closed grey). FIG. 10e, 30 minutes activation of ILC3 by GFL (open black) compared to vehicle BSA (open dash). Isotype (closed grey). FIG. 10f, 10 minutes activation of ILC3 by GFL. pERK n=8; pAKT n=8; phosphorylated p38/MAP kinase n=8; pSTAT3 n=8. Similar results were obtained in at least 3-4 independent experiments. Error bars show s.e.m.*P<0.05; **P<0.01; ns not significant.

FIGS. 11a-11c. Alterations in the diversity of intestinal commensal bacteria of Ret^{Δ} mice. FIG. 11a, Quantitative PCR analysis at the Phylum level in stool bacterial from co-housed Ret^{fl} and Ret^{Δ} littermates in steady state. n=5. FIG. 11b, Metagenomic Phylum level comparisons in stool bacterial from co-housed Ret^{fl} and Ret^{Δ} littermates in steady state (left) and after DSS treatment (right). n=5. FIG. 11c, Genus level comparisons in stool bacterial from co-housed Ret^{fl} and Ret^{Δ} littermates in steady state (left) and after DSS treatment (right). n=5. Error bars show s.e.m.*P<0.05; **P<0.01; ns not significant.

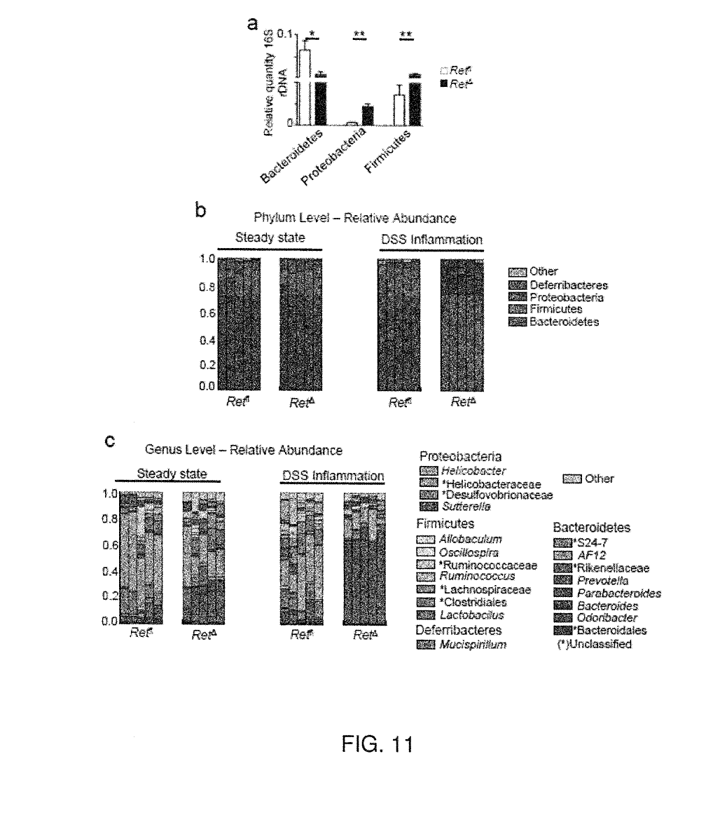

FIGS. 12a-12g. GFL expressing glial cells anatomically co-localise with ILC3. FIG. 12a, Intestine of Ret^{GFP} mice. Green: RET/GFP; Red: GFAP; Blue: RORγt. Similar results were obtained in three independent experiments. FIG. 12b, Purified lamina propria LTi, NCR⁻ and NCR⁺ILC3 subsets, T cells (T), B cells (B), Dendritic cells (Dc), Macrophages (Mø), enteric Neurons (N) and mucosal Glial cells (G). FIG. 12c, Neurosphere-derived glial cells. FIG. 12d, M: medium. Activation of neurosphere-derived glial cells with TLR2 (Pam3CSK4), TLR3 (Poli I:C), TLR4 (LPS) and TLR9 (DsDNA-EC) ligands, as well as IL-1β, IL-18 and IL-33. n=6. FIG. 12e, Il22 in co-cultures of glial and ILC3 using single or combined GFL antagonists. n=6. FIG. 12f, Il22 in co-cultures of ILC3 and glial cells from Illb^{-/-} or their WT controls. n=3. FIG. 12g, Gdnf Artn and Nrtn expression in glial cells and ILC3 upon TLR2 stimulation. n=3. Scale bar: 30 μm. Similar results were obtained in at least 4 independent experiments.

FIGS. 13a-13h. Glial cell sensing via MYD88 signals. a-c, Intestinal glial cells were purified by flow cytometry. FIG. 13a, Germ-free (GF) and their respective Specific Pathogen Free (SPF) controls. n=3. FIG. 13b, Myd88^{-/-} and their respective WT littermate controls. n=3. c, Gfap-Cre.Myd88^{Δ} and their littermate controls (Myd88^{fl}). n=3. FIG. 13d, Total lamina propria cells of Gfap-Cre.Myd88^{Δ} and their littermate controls (Myd88^{fl}). n=6. FIGS. 13e-13h, Citrobacter rodentium infection of Gfap-Cre.Myd88^{Δ} mice and their littermate controls (Myd88^{fl}). n=6. FIG. 13e, Innate IL-22. FIG. 13f, Citrobacter rodentium translocation. FIG. 13g, Infection burden. FIG. 13h, Weight loss. Data are representative of 3 independent experiments. Error bars show s.e.m.*P<0.05; **P<0.01; ns not significant.

FIG. 14. A novel glial-ILC3-epithelial cell unit orchestrated by neurotrophic factors. Lamina propria glial cells sense microenvironmental products, that control neurotrophic factor expression. Glial-derived neurotrophic factors operate in an ILC3-intrinsic manner by activating the tyrosine kinase RET, which directly regulates innate IL-22 downstream of a p38 MAPK/ERK-AKT cascade and STAT3 phosphorylation. GFL induced innate IL-22 acts on epithelial cells to induce reactivity gene expression (CBP: Commensal bacterial products; AMP: antimicrobial peptides; Muc: mucins). Thus, neurotrophic factors are the molecular link between glial cell sensing, innate IL-22 production and intestinal epithelial barrier defence.

### DETAILED DESCRIPTION

Group 3 innate lymphoid cells (ILC3) produce pro-inflammatory cytokines, regulate mucosal homeostasis and anti-microbial defence¹. In addition to their well-established developmentally regulated program, ILC3 are also controlled by microbial and dietary signals¹⁻⁶ raising the hypothesis that ILC3 possess other unexpected environmental sensing strategies. Neurotrophic factors are extra-cellular environmental cues to neurons and include the glial-derived neurotrophic factor (GDNF) family ligands (GFL) that activate the tyrosine kinase receptor RET in the nervous system, kidney and haematopoietic progenitors⁷⁻¹¹.

As demonstrated the data shown herein, in addition to their well-established capacity to integrate dendritic cell-derived cytokines¹, ILC3 perceive distinct multi-tissue regulatory signals leading to STAT3 activity and IL-22 expression, notably via integration of glial cell-derived neuroregulators. Thus, rather than providing hard-wired signals for ILC3-immunity, RET signals critically fine-tune innate IL-22 leading to efficient gut homeostasis and defence.

Previous studies demonstrated that neurons can indirectly shape foetal lymphoid tissue inducer cells and that ablation of glial cells leads to gut inflammation^{28,29}. As described herein, glial cells are central hubs of neuronal and innate immune regulation. Notably, neurotrophic factors are the molecular link between glial cell sensing, innate IL-22 and intestinal epithelial defence. Thus, glial/immune cell units might be also critical to the homeostasis of other barriers, notably in the skin, lung and brain³⁰. From an evolutionary perspective, coordination of innate immunity and neuronal function may ensure efficient mucosal homeostasis and a co-regulated neuro-immune response to various environmental challenges, including xenobiotics, intestinal infection, dietary aggressions and cancer.

### Increasing Activity of ILC3

The methods disclosed herein include methods for increasing production of interleukin-22 (IL-22) by Group 3 innate lymphoid cells (ILC3s) by contacting ILC3 with an agonist of RET in an amount effective to increase production of IL-22.

The methods disclosed herein also include methods for treating a disease associated with Group 3 innate lymphoid cells (ILC3) by administering to a subject in need of such treatment an agonist of RET in an amount effective to treat the disease.

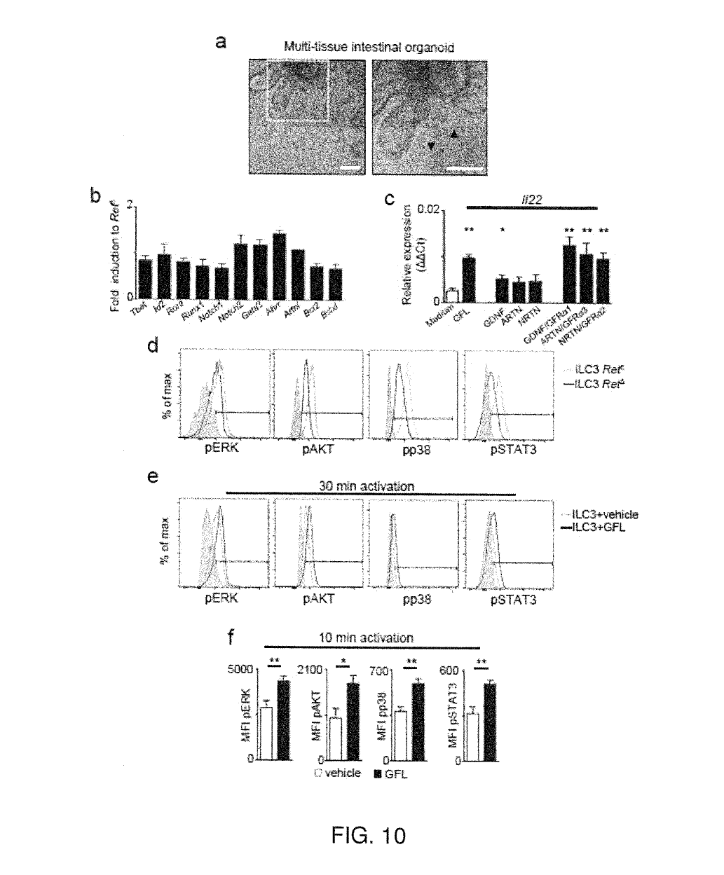

Other methods for treating disease include administering to a subject in need of such treatment a composition comprising activated ILC3 in an amount effective to treat the disease. In some of these methods, the composition comprising activated ILC3 also includes an agonist of RET. Alternatively, an agonist of RET can be administered separately from the composition comprising activated ILC3. As described herein, ILC3 can be activated by contacting ILC3 with one or more GDNF family ligand (GFL)/GDNF Family binding Receptor alpha (GFRα) pairs. Activation using one or all of GDNF/GFRα1, ARTN/GFRα3 and NRTN/GFRα2 are shown in FIG. 10c; other combinations of these pairs, and PSPN/GFRα4 alone or combined with other GFL/GFRα pairs also can be used.

Also provided herein are agonists of RET for use in treating a disease associated with ILC3, and compositions comprising activated ILC3 (and optionally an agonist of RET) for use in treating a disease associated with ILC3.

As used herein, RET (rearranged during transfection) is a receptor tyrosine kinase for members of the glial cell line-derived neurotrophic factor (GDNF) family of extracellular signaling molecules, and is also known as Ret, PTC, RET51, RET9, c-Ret, CDHF12, CDHR16, HSCR1, MEN2A, MEN2B, MTC1, RET-ELE1, and ret proto-oncogene. The amino acid sequence can be found at, e.g., UniProtKB P07949; it has two isoforms, P07949-1 (isoform 1) and P07949-2 (isoform 2). The nucleotide sequence can be found at, e.g., X15262 (mRNA/cDNA sequence).

As described elsewhere herein, an agonist of RET includes (1) a combination of a soluble GDNF Family binding Receptor alpha (GFRα) and a GFRα ligand (GFL) or an analog or mimetic thereof; or (2) an antibody that specifically binds to RET and increases RET tyrosine kinase activity or an antigen-binding fragment thereof.

Contacting ILC3 with an agonist of RET can be performed in vitro, or can be performed in vivo. In some embodiments of methods in which the contacting of ILC3 with an agonist of RET is performed in vivo, the agonist of RET is administered to a subject, such as a human. In some of these methods, the subject is not otherwise in need of treatment with the agonist of RET.

In the disclosed methods, the subject can be a human. In some of these methods, the subject is not otherwise in need of treatment with the agonist of RET and/or treatment with the ILC3.

Diseases treatable by the disclosed methods include infection, inflammation, neoplasia including colorectal cancer, and altered gut physiology.

The agonist of RET and/or the activated ILC3 can be administered by any suitable route of administration or delivery method. Suitable routes of administration include intravenous, oral, nasal, rectal or through skin absorption.

The agonist of RET and/or the activated ILC3 can be administered at any suitable interval, including daily, twice daily, three times per day, four times per day, every other day, weekly, every two weeks, every four weeks, continuously (e.g., by infusion, patch, or pump), and so on.

### Decreasing Activity of ILC3

Additional methods disclosed herein include methods for decreasing production of interleukin-22 (IL-22) by Group 3 innate lymphoid cells (ILC3) by contacting ILC3 with an antagonist of RET in an amount effective to decrease production of IL-22 by the ILC3.

The methods disclosed herein also include methods for treating a disease associated with Group 3 innate lymphoid cells (ILC3) by administering to a subject in need of such treatment an antagonist of RET in an amount effective to treat the disease.

Also provided herein are antagonists of RET for use in treating a disease associated with ILC3.

As described elsewhere herein, an antagonist of RET includes an inhibitory nucleic acid molecule that reduces that reduces expression, transcription or translation of RET, such as a sRNA, shRNA, or antisense nucleic acid molecule; an antibody that specifically binds and inhibits RET or an antigen-binding fragment thereof, or a small molecule antagonist of RET.

Contacting ILC3 with an antagonist of RET can be performed in vitro, or can be performed in vivo. In some embodiments of methods in which the contacting of ILC3 with an antagonist of RET is performed in vivo, the antagonist of RET is administered to a subject, such as a human. In some of these methods, the subject is not otherwise in need of treatment with the antagonist of RET.

In the disclosed methods, the subject can be a human. In some of these methods, the subject is not otherwise in need of treatment with the antagonist of RET.

In the methods disclosed herein for treating disease by administering an antagonist of RET, the disease can be epithelial intestinal cancer.

The antagonist of RET can be administered by any suitable route of administration or delivery method. Suitable routes of administration include intravenous, oral, nasal, rectal or through skin absorption.

The antagonist of RET can be administered at any suitable interval, including daily, twice daily, three times per day, four times per day, every other day, weekly, every two weeks, every four weeks, continuously (e.g., by infusion, patch, or pump), and so on.

### Agonists of Rearranged During Transfection (RET)

Agonists of RET include (1) a combination of a soluble GDNF Family binding Receptor alpha (GFRα) and a GFRα ligand (GFL) or an analog or mimetic thereof; or (2) antibodies that specifically bind to RET and increase RET tyrosine kinase activity or an antigen-binding fragment thereof. The agonists of RET may directly affect the tyrosine kinase activity of RET, or may increase or induce RET dimerization, with a resultant increase of RET tyrosine kinase activity.

The RET agonists may be entirely specific for RET, may agonize RET preferentially (as compared to other tyrosine kinases), or may agonize both RET and other tyrosine kinases. Such agonists may be useful even if RET is agonized less than other tyrosine kinases, but it is preferred that the agonists used in the methods described herein agonize RET to a greater extent than other tyrosine kinases. As used herein agonizing RET preferentially (as compared to other tyrosine kinases) means that the agonist agonizes RET at least 10%, 25%, 50%, 100%, 200%, 300%, 400%, 500%, 600%, 700%, 800%, 900%, 1000%, or more than other tyrosine kinases.

The combination of a soluble GFRα and a GFRα ligand (GFL) or an analog or mimetic thereof includes: (1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or (2) a combination of two or more of (a), (b), (c), (d), (e), (f) and (g).

Soluble GFRα molecules and GFRα ligands (GFLs) include the GFRαs and GFLs described herein, e.g., GFRα1, GFRα2, GFRα3, and GFRα4; and respective ligands GDNF, NTRN, ATRN, and PSPN. Analogs, mimetics, derivatives, and conjugates of GFRαs and GFLs include GFRα and GFL analogs having variations in amino acid sequence relative to natural GFRα and GFL sequences but which retain function of activating RET.

GFRα1 is also known as GDNF receptor, GFRA1, GDNFR, GDNFRA, GFR-ALPHA-1, RET1L, RETL1, TRNR1, and GDNF family receptor alpha 1. The amino acid sequence can be found at, e.g., UniProtKB P56159; it has two isoforms, P56159-1 (isoform 1) and P56159-2 (isoform 2). The nucleotide sequence can be found at, e.g., AF042080.1 (mRNA/cDNA sequence).

GFRα2 is also known as neurturin receptor, GFRA2, GDNFRB, NRTNR-ALPHA, NTNRA, RETL2, TRNR2, and GDNF family receptor alpha 2. The amino acid sequence can be found at, e.g., UniProtKB-000451; it has three isoforms, 000451-1 (isoform 1), 000451-2 (isoform 2) and 000451-3 (isoform 3). The nucleotide sequence can be found at, e.g., AY326396 (mRNA/cDNA sequence).

GFRα3 is also known as artemin receptor, GFRA3, GDNFR3, and GDNF family receptor alpha. The amino acid sequence can be found at, e.g., UniProtKB 060609; it has two isoforms, 060609-1 (isoform 1) and 060609-2 (isoform 2). The nucleotide sequence can be found at, e.g., AK297693 (mRNA/cDNA sequence).

GFRα4 is also known as persephin receptor and GFRA4. The amino acid sequence can be found at, e.g., UniProtKB Q9GZZ7; it has three isoforms, Q9GZZ7-1 (isoform GFRα1pha4b), Q9GZZ7-2 (isoform GFRα1pha4a) and Q9GZZ7-3 (isoform GFRα1pha4c). The nucleotide sequence can be found at, e.g., AF253318.

Glial cell-derived neurotrophic factor is also known as GDNF, ATF1, ATF2, HFB1-HSCR3, and glial cell derived neurotrophic factor. The amino acid sequence can be found at, e.g., UniProtKB P39905; it has three isoforms, P39905-1 (isoform 1), P39905-2 (isoform 2) and P39905-3 (isoform 3), P39905-2 (isoform 4) and P39905-3 (isoform 5). The nucleotide sequence can be found at, e.g., CR541923 (mRNA/cDNA sequence).

Neurturin is also known as NTRN. The amino acid sequence can be found at, e.g., UniProtKB Q99748. The nucleotide sequence can be found at, e.g., BC137399 (mRNA/cDNA sequence).

Artemin is also known as ATRN, enovin, neublastin, EVN and NBN. The amino acid sequence can be found at, e.g., UniProtKB Q5T4W7; it has three isoforms, Q5T4W7-1 (isoform 1), Q5T4W7-2 (isoform 2) and Q5T4W7-3 (isoform 3). The nucleotide sequence can be found at, e.g., AF109401 (mRNA/cDNA sequence).

Persephin is also known as PSPN. The amino acid sequence can be found at, e.g., UniProtKB 060542. The nucleotide sequence can be found at, e.g., AF040962 (mRNA/cDNA sequence).

Examples of analogs, derivatives, and conjugates of GFLs include: the variants of GDNF which retain an GDNF receptor agonist function described in U.S. Pat. No. 9,133,441; the variants of GDNF described in U.S. Pat. No. 9,243,046; the GFL variants (e.g. ΔN-GDNF) that efficiently activate RET but lack heparin-binding sites and do not interact with HSPGs in extracellular matrix described in U.S. Pat. No. 8,034,572; the neurturin molecules that have reduced heparin, heparan sulfate and heparan sulfated proteoglycan binding ability but retain the ability to induce phosphorylation of the RET protein described in U.S. Pat. Nos. 8,445,432, 9,127,083 and 9,469,679; the GDNF derived peptides described in U.S. Pat. No. 8,138,148; the neublastin molecules and dimerized proteins described in U.S. Pat. Nos. 7,276,580, 7,598,059 and 7,655,463; and the chimeric GDNF family ligands which activate GFRα/RET described in U.S. Pat. No. 6,866,851.

Other examples of analogs, derivatives, and conjugates of GFLs include: the GDNF analogs described in WO 2012/151476, EP 2440581, and other patent publications referenced therein, isoforms, precursors, fragments and splice variants of GDNF, such as those described in WO 2009/053536, US 2009/0069230, WO 2008/069876, WO 2007/019860, and US 2006/0258576.

Still other agonists of RET include the GDNF family ligands (GFL) and mimetics or RET signaling pathway activators and direct RET activators described in U.S. Pat. No. 8,901,129.

Another agonist of RET is a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035). As shown by Tokugawa et al. (Neurochem Int. 2003 January; 42(1):81-6), XIB4035, like GDNF, induced RET autophosphorylation. A chemical structure of XIB4035 is shown below:

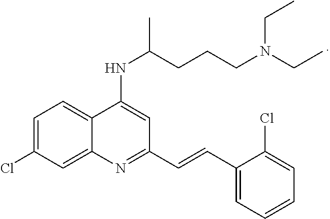 [SMILES: CCN(CC)CCCC(C)Nc1cc(/C=C/c2ccccc2Cl)nc2cc(Cl)ccc12]

Another agonist of RET is a soluble GFRα and a BT compound. BT compounds are described in WO 2011/070177.

Another agonist of RET is a soluble GFRα and an antibody that specifically binds to and dimerizes the GFRα. Antibodies that specifically bind to a GFRα and dimerize the GFRα can be obtained by screening for this activity among a set of GFRα-binding antibodies.

Additional agonists of RET are antibodies that specifically bind to RET and increase RET tyrosine kinase activity or an antigen-binding fragment of such antibodies. RET-binding antibodies are known in the art, such as those described in U.S. Pat. No. 6,861,509, and various commercially-available antibodies. Antibodies that specifically bind to RET and increase RET tyrosine kinase activity can be obtained by screening for this activity among a set of RET-binding antibodies.

### Antagonists of RET

Antagonists of RET include peptide antagonists (including modified peptides and conjugates), inhibitory antibody molecules, inhibitory nucleic acid molecules, and small molecules. Some of the RET antagonists may be entirely specific for RET, may antagonize RET preferentially (as compared to other tyrosine kinases), or may antagonize both RET and other tyrosine kinases (such as some of the small molecule RET tyrosine kinase inhibitors described below. Such antagonists may be useful even if RET is antagonized less than other tyrosine kinases, but it is preferred that the antagonists used in the methods described herein antagonize RET to a greater extent than other tyrosine kinases. As used herein, antagonizing RET preferentially (as compared to other tyrosine kinases) means that the antagonist antagonizes RET at least 10%, 25%, 50%, 100%, 200%, 300%, 400%, 500%, 600%, 700%, 800%, 900%, 1000%, or more than other tyrosine kinases.

Antagonists of RET include antibodies that specifically bind and inhibit: (a) RET tyrosine kinase activity, (b) a GDNF Family binding Receptor alpha (GFRα), or (c) a GFRα ligand, or an antigen-binding fragment thereof. Examples include the antibodies described in U.S. Pat. Nos. 8,968,736, 9,522,185, and US 2017/0096488 that bind human GFRα3. RET-binding antibodies are known in the art, such as those described in U.S. Pat. No. 6,861,509, and various commercially-available antibodies. Antibodies that specifically bind to and inhibit: (a) RET tyrosine kinase activity, (b) a GDNF Family binding Receptor alpha (GFRα), or (c) a GFRα ligand, can be obtained by screening for one of these activities among a set of antibodies binding to RET, a GFRα, or a GFRα ligand.

Antagonists of RET include an inhibitory nucleic acid molecule that reduces expression, transcription or translation of RET, a GFRα, or a GFRα ligand. Suitable inhibitory nucleic acid molecules include: RET-specific, a GFRα-specific, or a GFRα ligand-specific inhibitory nucleic acid, e.g., an siRNA, antisense, aptamer, or ribozyme targeted specifically to RET, a GFRα, or a GFRα ligand.

Antagonists of RET include a RET tyrosine kinase inhibitor. Exemplary RET tyrosine kinase inhibitors include AST 487, motesanib, cabozantinib, vandetanib, ponatinib, sunitinib, sorafenib, and alectinib.

AST 487 (also known as NVP-AST487; 630124-46-8; UNII-W34UO2M4T6); IUPAC name: 1-[4-[(4-ethylpiperazin-1-yl)methyl]-3-(trifluoromethyl)phenyl]-3-[4-[6-(methylamino)pyrimidin-4-yl]oxyphenyl]urea) is an inhibitor of RET, receptor-type tyrosine-protein kinase FLT3, Kinase Insert Domain Receptor (KDR; VEGFR2), Abelson murine leukemia viral oncogene homolog 1 (c-ABL), and stem cell factor receptor (c-KIT) that has been shown to inhibit RET autophosphorylation and activation of downstream effectors (Akeno-Stuart et al., Cancer Res. 2007 Jul. 15; 67(14):6956-64). A chemical structure of AST 487 is shown below:

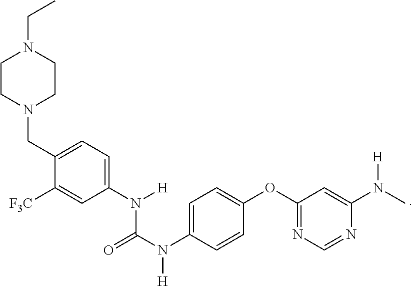 [SMILES: CCN1CCN(Cc2ccc(NC(=O)Nc3ccc(Oc4cc(NC)ncn4)cc3)cc2C(F)(F)F)CC1]

Motesanib (also known as AMG-706; IUPAC name: N-(3,3-dimethyl-2,3-dihydro-1H-indol-6-yl)-2-[(pyridin-4-ylmethyl)amino]pyridine-3-carboxamide) is an inhibitor of RET, VEGFRs, platelet-derived growth factor receptors (PDGFRs), and c-KIT. A chemical structure of motesanib is shown below:

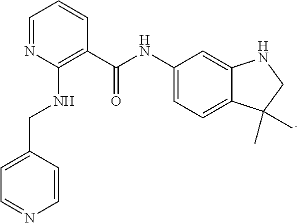 [SMILES: CC1(C)CNc2cc(NC(=O)c3cccnc3NCc3ccncc3)ccc21]

Cabozantinib (also known as CABOMETYX; COMETRIQ; XL-184; BMS-907351; IUPAC name: N-(4-((6,7-dimethoxyquinolin-4-yl)oxy)phenyl)-N′-(4-fluorophenyl)cyclopropane-1,1-dicarboxamide) is an inhibitor of RET, hepatocyte growth factor receptor (MET), AXL receptor tyrosine kinase (AXL; tyrosine-protein kinase receptor UFO) and vascular endothelial growth factor receptor receptors (VEGFR) including VEGFR2. A chemical structure of cabozantinib is shown below:

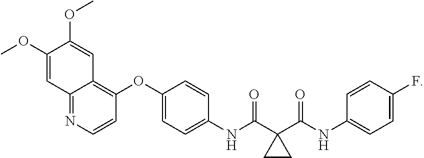 [SMILES: COc1cc2nccc(Oc3ccc(NC(=O)C4(C(=O)Nc5ccc(F)cc5)CC4)cc3)c2cc1OC]

Vandetanib (also known as CAPRELSA; ZACTIMA; ZD-6474; IUPAC name: N-(4-bromo-2-fluorophenyl)-6-methoxy-7-((1-methylpiperidin-4-yl)methoxy)quinazolin-4-amine) is an inhibitor of RET, VEGFRs including VEGFR2, and epidermal growth factor receptor (EGFR). A chemical structure of vandetanib is shown below:

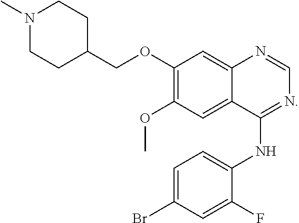 [SMILES: COc1cc2c(Nc3ccc(Br)cc3F)ncnc2cc1OCC1CCN(C)CC1]

Ponatinib (also known as ICLUSIG; AP24534; IUPAC name: 3-(2-Imidazo[1,2-b]pyridazin-3-ylethynyl)-4-methyl-N-[4-[(4-methylpiperazin-1-yl)methyl]-3-(trifluoromethyl)phenyl]benzamide) is an inhibitor of RET and fibroblast growth factor receptor (FGFR). A chemical structure of ponatinib is shown below:

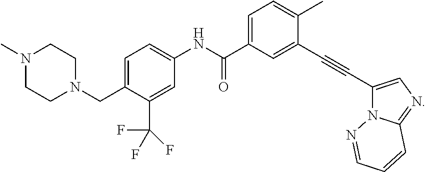 [SMILES: Cc1ccc(C(=O)Nc2ccc(CN3CCN(C)CC3)c(C(F)(F)F)c2)cc1C#Cc1cnc2cccnn12]

Sunitinib (also known as SUTENT; SU11248; IUPAC name: N-(2-diethylaminoethyl)-5-[(Z)-(5-fluoro-2-oxo-1H-indol-3-ylidene)methyl]-2,4-dimethyl-1H-pyrrole-3-carboxamide) is an inhibitor of RET, PGFRs, VEGFRs, c-KIT, granulocyte colony-stimulating factor receptor (GCSFR) and FLT3. A chemical structure of sunitinib is shown below:

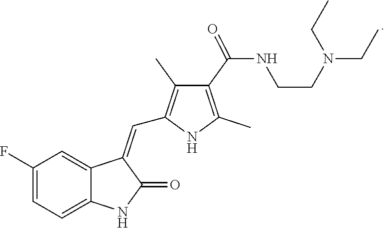 [SMILES: CCN(CC)CCNC(=O)c1c(C)[nH]c(/C=C2\C(=O)Nc3ccc(F)cc32)c1C]

Sorafenib (also known as NEXAVAR; IUPAC name: 4-[4-[[4-chloro-3-(trifluoromethyl)phenyl]carbamoylamino] phenoxy]-N-methyl-pyridine-2-carboxamide) is an inhibitor of RET, VEGFR, PDGFR and Raf family kinases. A chemical structure of sorafenib is shown below:

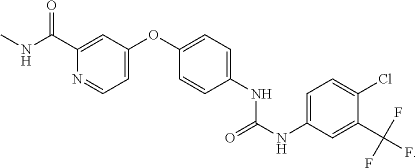 [SMILES: CNC(=O)c1cc(Oc2ccc(NC(=O)Nc3ccc(Cl)c(C(F)(F)F)c3)cc2)ccn1]

Alectinib (also known as ALECENSA; IUPAC name: 9-ethyl-6,6-dimethyl-8-[4-(morpholin-4-yl)piperidin-1-yl]-11-oxo-6,11-dihydro-5H-benzo[b]carbazole-3-carbonitrile) is an inhibitor of RET, and anaplastic lymphoma kinase (ALK). A chemical structure of alectinib is shown below:

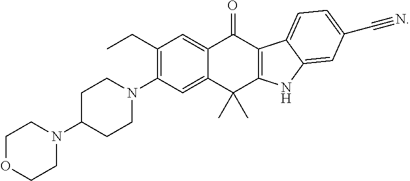 [SMILES: CCc1cc2c(cc1N1CCC(N3CCOCC3)CC1)C(C)(C)c1[nH]c3cc(C#N)ccc3c1C2=O]

Other suitable RET antagonists include the molecules described in: U.S. Pat. Nos. 6,235,769, 7,504,509, 8,067,434, 8,426,437, 8,629,135, 8,937,071, 8,999,973, 9,035,063, 9,382,238, 9,297,011, US 2015/0238477, US 2015/0272958, US 2016/0271123, US 20160354377, US 2017/0096425, and US 2017/0121312, and related patent applications worldwide.

A subject shall mean a human or vertebrate mammal including but not limited to a dog, cat, horse, goat and non-human primate, e.g., monkey. Preferably the subject is a human. In some embodiments the subject is one who is not otherwise in need of treatment with an RET agonist or RET antagonist. Therefore the subject, in specifically identified embodiments, may be one who has not been previously diagnosed with a disorder for which an RET agonist or RET antagonist is an identified form of treatment.

The subject can be first identified as a subject in need of treatment, such as one having a disease that is treatable by the methods disclosed herein, and then treated with an RET agonist (and/or ILC3) or RET antagonist. The skilled artisan is aware of methods for identifying a subject as having a disease that is treatable by the methods disclosed herein.

As used herein, the terms “treat,” “treated,” or “treating” refers to a treatment of a disease that ameliorates the disease (disease modification), ameliorates symptoms of the disease, prevents the disease from becoming worse, or slows the progression of the disease compared to in the absence of the therapy.

A “disease associated with Group 3 innate lymphoid cells (ILC3)” as used herein is a disease or disorder in which ILC3 play some role in the development, maintenance or worsening of the disease or disorder.

In some of the methods disclosed herein, such diseases can be effectively treated by increasing production of IL-22 by ILC3, such as by contacting ILC3 with an agonist of RET in an amount effective to increase production of IL-22 by the ILC3; by administering to a subject in need of such treatment an agonist of RET in an amount effective to treat the disease; or by administering ILC3 (and optionally an agonist of RET) in an amount effective to treat the disease.

Diseases treatable by such methods include: infection, inflammation, neoplasia including colorectal cancer, and altered gut physiology.

In other of the methods disclosed herein, the diseases can be effectively treated by decreasing production of IL-22 by ILC3, such as by contacting ILC3 with an antagonist of RET in an amount effective to decrease production of IL-22 by the ILC3; or by administering to a subject in need of such treatment an antagonist of RET in an amount effective to treat the disease.

Diseases treatable by such methods include: epithelial intestinal cancer.

Toxicity and efficacy of the methods of the present invention can be determined by standard pharmaceutical procedures in cell cultures or experimental animals, e.g., for determining the LD₅₀ (the dose lethal to 50% of the population) or TD₅₀ (the dose toxic to 50% of the population) and the ED₅₀ (the dose therapeutically effective in 50% of the population). The dose ratio between toxic and therapeutic effects is the therapeutic index and it can be expressed as the ratio LD₅₀/ED₅₀ or TD₅₀/ED₅₀. Therapeutic agents that exhibit large therapeutic indices are preferred. While therapeutic agents that exhibit toxic side effects may be used, in such cases it is preferred to use a delivery system that targets such agents to the site of affected tissue in order to minimize potential damage to other cells or tissues and, thereby, reduce side effects.

The data obtained from the cell culture assays and/or animal studies can be used in formulating a range of dosage of the therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Other, higher percentages of an active compound also can be used.

The pharmaceutical compositions may also be, and preferably are, sterile in some embodiments. In other embodiments the compounds may be isolated. As used herein, the term “isolated” means that the referenced material is removed from its native environment, e.g., a cell. Thus, an isolated biological material can be free of some or all cellular components, i.e., components of the cells in which the native material is occurs naturally (e.g., cytoplasmic or membrane components). In the case of nucleic acid molecules, an isolated nucleic acid includes a PCR product, an isolated RNA, a synthetically (e.g., chemically) produced RNA, such as an siRNA, an antisense nucleic acid, an aptamer, etc. Isolated nucleic acid molecules include sequences inserted into plasmids, cosmids, or other vectors to form part of a chimeric recombinant nucleic acid construct, or produced by expression of a nucleic acid encoding it. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein, or may be synthetically (e.g., chemically) produced, or produced by expression of a nucleic acid encoding it. An isolated cell, such as an ILC3 cell, can be removed from the anatomical site in which it is found in an organism, or may be produced by in vitro expansion of an isolated cell or cell population. An isolated material may be, but need not be, purified.

The term “purified” in reference to a protein, a nucleic acid, or a cell or cell population, refers to the separation of the desired substance from contaminants to a degree sufficient to allow the practitioner to use the purified substance for the desired purpose. Preferably this means at least one order of magnitude of purification is achieved, more preferably two or three orders of magnitude, most preferably four or five orders of magnitude of purification of the starting material or of the natural material. In specific embodiments, a purified agonist of RET or antagonist of RET or ILC3 population is at least 60%, at least 80%, or at least 90% of total protein or nucleic acid or cell population, as the case may be, by weight. In a specific embodiment, a purified agonist of RET or antagonist of RET or ILC3 population is purified to homogeneity as assayed by standard, relevant laboratory protocols.

In some embodiments a purified and or isolated molecule is a synthetic molecule.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 1 mg/kg/body weight to about 100 mg/kg/body weight, about 5 microgram/kg/body weight to about 500 milligram/kg/body weight, etc., can be administered, based on the numbers described above. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment. Multiple doses of the molecules of the invention are also contemplated.

The compounds and/or cells described herein may be used alone without other active therapeutics or may be combined with other therapeutic compounds for the treatment of the diseases described herein.

When used in combination with the compounds and cells described herein, the dosages of known therapies may be reduced in some instances, to avoid side effects. In some instances, when the compounds and/or cells described herein are administered with another therapeutic, a sub-therapeutic dosage of either the compounds and/or cells described herein or the known therapies, or a sub-therapeutic dosage of both, is used in the treatment of a subject. A “sub-therapeutic dose” as used herein refers to a dosage which is less than that dosage which would produce a therapeutic result in the subject if administered in the absence of the other agent. Thus, the sub-therapeutic dose of a known therapy is one which would not produce the desired therapeutic result in the subject in the absence of the administration of the compounds and cells described herein. Existing therapies for the diseases described herein are well known in the field of medicine, and may be described in references such as Remington's Pharmaceutical Sciences; as well as many other medical references relied upon by the medical profession as guidance for treatment.

When the compounds and/or cells described herein are administered in combination with other therapeutic agents, such administration may be simultaneous or sequential. When the other therapeutic agents are administered simultaneously they can be administered in the same or separate formulations, but are administered at the same time. The administration of the other therapeutic agent and the compounds and/or cells described herein can also be temporally separated, meaning that the other therapeutic agents are administered at a different time, either before or after, the administration of the compounds and cells described herein. The separation in time between the administration of these compounds may be a matter of minutes or it may be longer.

The active agents of the invention (e.g., the compounds and cells described herein) are administered to the subject in an effective amount for treating disease. According to some aspects of the invention, an effective amount is that amount, depending on the disease being treated, of a RET agonist (and/or ILC3) or RET antagonist alone or in combination with another medicament, which when combined or co-administered or administered alone, results in a therapeutic response to the disease. The biological effect may be the amelioration and or absolute elimination of disease, or of symptoms resulting from the disease. In another embodiment, the biological effect is the complete abrogation of the disease, as evidenced for example, by the absence of a symptom of the disease.

The effective amount of a compound (i.e., any of the agonists, antagonists, or ILC3) used in methods of the invention in the treatment of a disease described herein may vary depending upon the specific compound used, the mode of delivery of the compound, and whether it is used alone or in combination. The effective amount for any particular application can also vary depending on such factors as the disease being treated, the particular compound being administered, the size of the subject, or the severity of the disease or condition. One of ordinary skill in the art can empirically determine the effective amount of a particular molecule of the invention using routine and accepted methods known in the art, without necessitating undue experimentation. Combined with the teachings provided herein, by choosing among the various active compounds and weighing factors such as potency, relative bioavailability, patient body weight, severity of adverse side-effects and preferred mode of administration, an effective therapeutic treatment regimen can be planned which does not cause substantial toxicity and yet is effective to treat the particular subject.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by relevant government regulatory agencies. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

The therapeutic compositions used as described herein may comprise different types of carriers depending on whether it is to be administered in solid, liquid or aerosol form, and whether it need to be sterile for such routes of administration as injection. The compounds and/or cells described herein can be administered intravenously, intradermally, intraarterially, intralesionally, intracranially, intraarticularly, intranasally, intravitreally, intravaginally, intrarectally, topically, intramuscularly, intraperitoneally, subcutaneously, intravesicularlly, mucosally, orally, locally, by inhalation (e.g., aerosol inhalation), by injection, by infusion including by continuous infusion, by localized perfusion, via a catheter, via a lavage, in cremes, in lipid compositions (e.g., liposomes), or by other method or any combination of the foregoing as would be known to one of ordinary skill in the art (see, for example, Remington's Pharmaceutical Sciences) and as is appropriate for the disease being treated.

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compounds described herein may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the compounds and/or cells described herein is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The compounds and/or cells described herein can be administered in various ways and to different classes of recipients. In some instances the administration is chronic. Chronic administration refers to long term administration of a drug to treat a disease. The chronic administration may be on an as needed basis or it may be at regularly scheduled intervals. For instance, the compounds and/or cells described herein may be administered twice daily, three times per day, four times per day, every other day, weekly, every two weeks, every four weeks, continuously (e.g., by infusion, patch, or pump), and so on.

The compounds and/or cells described herein may be administered directly to a tissue. Direct tissue administration may be achieved by direct injection. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The compounds and/or cells described herein are administered in pharmaceutically acceptable solutions, which may routinely contain pharmaceutically acceptable concentrations of salt, buffering agents, preservatives, compatible carriers, adjuvants, and optionally other therapeutic ingredients.

According to the methods described herein, the compounds and/or cells described herein may be administered in a pharmaceutical composition. In general, a pharmaceutical composition comprises the compound of the invention and a pharmaceutically-acceptable carrier. Pharmaceutically-acceptable carriers useful with compounds and/or cells described herein are well-known to those of ordinary skill in the art. As used herein, a pharmaceutically-acceptable carrier means a non-toxic material that does not interfere with the effectiveness of the biological activity of the compounds and/or cells described herein.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds and/or cells described herein may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids, such as a syrup, an elixir or an emulsion.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds and/or cells described herein may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Remington's Pharmaceutical Sciences). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In yet other embodiments, vehicle for the compounds and/or cells described herein is a biocompatible microparticle or implant that is suitable for implantation into a mammalian recipient. Exemplary bioerodible implants are known in the art. The implant may be a polymeric matrix in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the compounds and/or cells described herein to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the compounds and/or cells described herein may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Other delivery systems can include time-release, delayed release or sustained release delivery systems. Such systems can avoid repeated administrations of the compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Such delivery systems also include non-polymer systems such as lipids including sterols such as cholesterol, cholesterol esters and fatty acids or neutral fats such as mono- di- and triglycerides; hydrogel release systems; silastic systems; peptide based systems; wax coatings; compressed tablets using conventional binders and excipients; partially fused implants; and the like. In addition, pump-based hardware delivery systems can be used, some of which are adapted for implantation.

Use of a long-term sustained release implant may be particularly suitable for treatment of chronic diseases. Long-term release, as used herein, means that the implant is constructed and arranged to delivery therapeutic levels of the active ingredient for at least 30 days, and preferably at least 60 days. Long-term sustained release implants are well-known to those of ordinary skill in the art and include some of the systems described above.

Thus the compounds and/or cells described herein described herein may, in some embodiments, be assembled into pharmaceutical or research kits to facilitate their use in therapeutic or research applications. A kit may include one or more containers housing the components of the invention and instructions for use. Specifically, such kits may include one or more compounds and/or cells described herein, along with instructions describing the intended therapeutic application and the proper administration of these agents. In certain embodiments the compounds and/or cells described herein in a kit may be in a pharmaceutical formulation and dosage suitable for a particular application and for a method of administration of the agents.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Materials and Methods

Mice: C57BL/6J mice were purchased from Charles River. Ret^{GFP 13}, Rag1^{-/-}γc^{-/- 31,32} Ret^{MEN2B 14}, Rosa26^{YFP 33}, Rosa26^{RFP 34}, Ret^{fl/fl 16}, Rorgt-Cre¹⁵, Illb^{-/-35} and Myd88^{-/-} 36 were in a full C57BL/6J background. Gfap-Cre²⁶ bred to Myd88^{fl/fl 27} were in F8-F9 to a C57B1/6J background. All lines were bred and maintained at IMM Lisboa animal facility. Mice were systematically compared with co-housed littermate controls. Both males and females were used in this study. Randomization and blinding were not used unless stated otherwise. All animal experiments were approved by national and institutional ethical committees, respectively Direção Geral de Veterinária and iMM Lisboa ethical committee. Germ-free mice were housed at Instituto Gulbenkian de Ciência, Portugal, and Institut Pasteur, France, in accordance to institutional guidelines for animal care. Power analysis was performed to estimate the number of experimental mice.

Generation of Foetal Liver Chimeras:

For reconstitution experiments, 5×10⁶ foetal liver cells were isolated from E14.5 Ret^{WT/GFP} or Ret^{GFP/GFP} mice and injected intravenously into non-lethally irradiated (200rad) alymphoid Rag1^{-/-}γc^{-/-} hosts. Mice were analysed 8 weeks post-transplantation.

Dextran Sodium Sulphate-Induced Colitis:

Dextran Sodium Sulphate (DSS) (molecular mass 36,000-50,000 Da; MP Biomedicals) was added into drinking water 3% (w/v) for 5 days followed by 2 days of regular water. Mice were analysed at day 7. Body weight, presence of blood and stool consistency was assessed daily.

Citrobacter rodentium Infection:

Infection with Citrobacter rodentium ICC 180 (derived from DBS 100 strain)³⁷ was performed by gavage inoculation of 10⁹ colony forming units^{37,38}. Acquisition and quantification of luciferase signal was performed in an IVIS system (Caliper Life Sciences). Throughout infection, weight loss, diarrhoea and bloody stools were monitored daily.

Antibiotic Treatment:

Pregnant females or new born mice were treated with streptomycin 5 g/L, ampicillin 1 g/L and colistin 1 g/L (Sigma-Aldrich) into drinking water with 3% sucrose. Control mice were given 3% sucrose in drinking water as previously described²².

Microscopy:

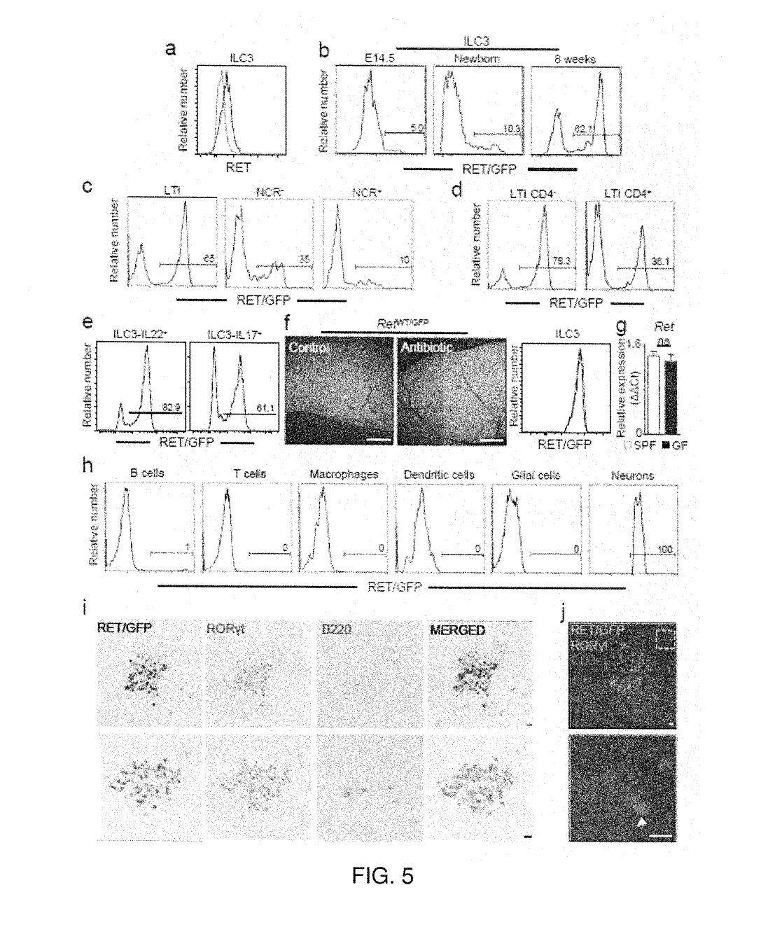

Intestines from Ret^{GFP} and Ret^{GFP} chimeras were imaged in a Zeiss Lumar V12 fluorescence stereo microscope with a NeoLumar S 0.8× objective using the GFP filter. Whole-mount analysis was performed as previously described^{2,9}. Briefly, adult intestines were flushed with cold PBS (Gibco) and opened longitudinally. Mucus and epithelium was removed and intestines were fixed in 4% PFA (Sigma-Aldrich) at room temperature for 10 minutes and incubated in blocking/permeabilising buffer solution (PBS containing 2% BSA, 2% goat serum, 0.6% Triton X-100). To visualise three-dimensional structures of the small intestine, samples were cleared with benzyl alcohol-benzyl benzoate (Sigma-Aldrich) prior dehydration in methanol^{2,9}. For analysis of thick gut sections intestines were fixed with 4% PFA at 4° C. overnight and were then included in 4% low-melting temperature agarose (Invitrogen). Sections of 100 μm were obtained with a Leica VT1200/VT1200 S vibratome and embedded in Mowiol (Calbiochem)². Slides or whole-mount samples were incubated overnight or for 1-2 days respectively at 4° C. using the following antibodies: rat monoclonal anti-B220 (RA3-6B2) (eBioscience), mouse monoclonal anti-RORγt (Q31-378) (BD Pharmigen), mouse monoclonal anti-GFAP (GA-5) (Sigma-Aldrich), mouse monoclonal anti-GFAP Cy3 (GA-5) (Abcam), anti-GDNF antibody (Abcam), DAPI (4′,6-Diamidino-2-Phenylindole, Dihydrochloride) (Invitrogen). A647 goat anti-rat, A568 goat anti-rat, A647 goat anti-mouse, A488 rabbit anti-GFP, and A488 goat anti-rabbit secondary antibodies were purchased from Invitrogen. Neurospheres and cultured glial cells were fixed in PFA 4% 10 minutes at room temperature and permeabilised in PBS-Triton 0.1% during 30 seconds. After several washing steps with PBS cells were incubated with antibodies during 3 h at room temperature and then mounted in Mowiol³⁹. Samples were acquired on a Zeiss LSM710 confocal microscope using EC Plan-Neofluar 10×/0.30 M27, Plan Apochromat 20×/0.8 M27 and EC Plan-Neofluar 40×/1.30 objectives. Three-dimensional reconstruction of images was achieved using Imaris software and snapshot pictures were obtained from the three-dimensional images. For analysis of confocal images, cells were counted using in-house software, written in MATLAB (Mathworks, Natick, Mass.). Briefly, single-cell ILC3 nuclei were identified via RORγt by thresholding and particle analysis. Regions of interest (ROIs) (FIG. 5i; Bottom panels) were defined from each nucleus for analysis in the GFP channel, where staining was considered positive if a minimum number of pixels (usually 20) were above a given threshold. The software allows for batch processing of multiple images and generates individual report images for user verification of cell-counting results and co-expression analysis: (https://imm.medicina.ulisboa.pt/en/servicos-e-recursos/technical-facilities/bioimaging).

Histopathology Analysis:

Colon samples were fixed in 10% neutral buffered formalin. The colon was prepared in multiple cross-sections or “Swiss roll” technique⁴⁰, routine-processed for paraffin embedding and 3-4 μm sections were stained with haematoxylin and eosin. Enteric lesions were scored by a pathologist blinded to experimental groups, according to previously published criteria⁴¹⁻⁴³. Briefly, lesions were individually scored (0-4 increasing severity) for the following criteria: 1-mucosal loss; 2-mucosal epithelial hyperplasia, 3-degree of inflammation, 4-extent of the section affected in any manner and 5-extent of the section affected in the most severe manner as previously described⁴³. Final scores were derived by summing the individual lesion and the extent scores. The internal diameter of the crypts was measured in at least five fields (10× magnification), corresponding to the hotspots in which the most severe changes in crypt architecture were seen. Measurements were performed in an average of 35 crypts per sample/mouse, from proximal to distal colon. Intestinal villus height was measured in the jejunum. Measurements were performed in slides scanned using a Hamamatsu Nanozoomer SQ digital slide scanner running NDP Scan software.

Enteric Glial Cell Isolation:

Enteric glial cells isolation was adapted from previously described protocols^{44,45}. Briefly, the muscularis layer was separated from the submucosa with surgical forceps under a dissection microscope (SteREO Lumar.V12, Zeiss). The lamina propria was scraped mechanically from the underlying submucosa using 1.5 mm cover-slips (Thermo Scientific). Isolated tissues were collected and digested with Liberase™ (7,5 μg/mL; Roche) and DNase I (0.1 mg/mL; Roche) in RPMI supplemented with 1% hepes, sodium pyruvate, glutamine, streptomycin and penicillin and 0.1% β-mercaptoethanol (Gibco) for approximately 40 min at 37° C. Single-cell suspensions were passed through a 100 μm cell strainer (BD Biosciences) to eliminate clumps and debris.

Flow Cytometry and Cell Sorting:

Lamina propria cells were isolated as previously described⁴⁶. Briefly, intestines were digested with collagenase D (0.5 mg/mL; Roche) and DNase I (0.1 mg/mL; Roche) in RPMI supplemented with 10% FBS, 1% hepes, sodium pyruvate, glutamine, streptomycin and penicillin and 0.1% β-mercaptoethanol (Gibco) for approximately 30 min at 37° C. under gentle agitation. For cytokine analysis, cell suspensions were incubated 4 h in PMA/Ionomycin (Sigma-Aldrich) and Brefeldin A (eBioscience) at 37° C. Intracellular staining was performed using IC fixation/permeabilisation kit (eBioscience). Cells were stained using PBS, 1% FBS, 1% hepes and 0.6% EDTA (Gibco). Flow cytometry analysis and cell sorting were performed using FORTESSA and FACSAria flow cytometers (BD Biosciences). Data analysis was done using FlowJo software (Tristar). Sorted populations were >95% pure. Cell suspensions were stained with anti-CD45 (30-F11), anti-TER119 (TER-119), TCRPβ (H57-597), anti-CD3ε (eBio500A2), anti-CD19 (eBio1D3), anti-NK1.1 (PK136), anti-CD11c (N418), anti-Gr1 (RB6-8C5), anti-CD11b (Mi/70), anti-CCR6 (29-2L17), anti-CD127 (IL-7Rα; A7R34), anti-Thy1.2 (53-2.1), anti-CD49b (DX5), anti-TCRδ (GL3), anti-NKp46 (29A1.4), anti-IL-17 (eBio17B7), anti-IL-22 (1H8PWSR), Rat IgG1 isotype control (eBRG1) antibodies, 7AAD viability dye, anti-Mouse CD16/CD32 (Fc block), anti-RORγt (AFKJS-9); Rat IgG2a_{κ} Isotype Control (eBR2a) and streptavidin fluorochrome conjugates all from eBioscience; anti-CD4 (GK1.5), anti-CD31 (390), anti-CD8α (53-6.7), anti-CD24 (M1/69), anti-Epcam (G8.8) antibodies were purchased from Biolegend. Anti-RET (IC718A) antibody was purchased from R&D Systems. LIVE/DEAD Fixable Aqua Dead Cell Stain Kit was purchased from Invitrogen. Cell populations were defined as: ILC3-CD45⁺Lin⁻Thy1.2^{hi}IL7Rα⁺RORγt⁺; For ILC3 subsets additional markers were employed: LTi-CCR6⁺Nkp46⁻; ILC3 NCR⁻-CCR6⁻Nkp46⁻; ILC3 NCR⁺-CCR6⁻ Nkp46⁺; Lineage was composed by CD3ε, CD8a, TCRP3, TCRγδ, CD19, Gr1, CD11c and TER119; Glial cells—CD45⁻CD31⁻TER119⁻CD49b⁺⁴⁷; T cells—CD45⁺CD3ε⁺; γδ T cells—CD45⁺CD3ε⁺γδTCR⁺; B cells —CD45⁺CD19⁺B220⁺; Macrophages—CD45⁺CD11b⁺F4/80⁺; Dendritic cells—CD45⁺CD19⁻CD3ε⁻MHCII⁺CD11c⁺; enteric neurons—CD45⁻RET/GFP⁺¹³Epithelial cells—CD45⁻CD24⁺Epcam⁺.

Quantitative RT-PCR:

Total RNA was extracted using RNeasy micro kit (Qiagen) or Trizol (Invitrogen) according to the manufacturer's protocol. RNA concentration was determined using Nanodrop Spectrophotometer (Nanodrop Technologies). Quantitative real-time RT-PCR was performed as previously described^{2,8,9}. Hprt and Gapdh were used as housekeeping genes. For TaqMan assays (Applied Biosystems) RNA was retro-transcribed using a High Capacity RNA-to-cDNA Kit (Applied Biosystems), followed by a pre-amplification PCR using TaqMan PreAmp Master Mix (Applied Biosystems). TaqMan Gene Expression Master Mix (Applied Biosystems) was used in real-time PCR. TaqMan Gene Expression Assays (Applied Biosystems) were the following: Gapdh Mm99999915_g1; Hprt Mm00446968_m1; Artn Mm00507845_m1; Nrtn Mm03024002_m1; Gdnf Mm00599849_m1; Gfra1 Mm00439086_m1; Gfra2 Mm00433584_m1; Gfra3 Mm00494589_m1; Ret Mm00436304_m1; Il22 Mm01226722_g; Il17a Mm00439618_m1; Il23r Mm00519943_m1; Rorgt Mm01261022_m1; Il7ra Mm00434295_m1; Ahr Mm00478932_m1; Stat3 Mm01219775_m1; Cxcr6 Mm02620517_s1; Nfkbiz Mm00600522_m1; RegIIIa Mm01181787_m1; RegIIIb Mm00440616_g1; RegIIIg Mm00441127_m1; Defa1 Mm02524428_g1; Defa-rs1 Mm00655850_m1; Defa5 Mm00651548_g1; Defa21 Mm04206099_gH; Muc1 Mm00449599_m1; Muc3 Mm01207064_m1; Muc13 Mm00495397_m1; Gfap Mm01253033_m1; Ascl2 Mm01268891_g; Tff3 Mm00495590_m1; Relm-b Mm00445845_m1; Pla2g2a Mm00448160_m1; Pla2g5 Mm00448162_m1; Wnt3 Mm00437336_m1; Ctnnb1 Mm00483039_m1; Axin2 Mm00443610_m1; Dll1b Mm01279269_m1; Il18 Mm00434225_m1; Tnfa Mm00443260_g1; Lyz1 Mm00657323_m1; Lrg5 Mm00438890_m1; Tbx21 Mm00450960_m1; Id2 Mm00711781_m1; Runx1 Mm01213404_m1; Notch1 Mm00435249_m1; Notch2 Mm00803077_m1; Gata3 Mm00484683_m1; Bcl2 Mm00477631_m1; Bcl2l1 Mm00437783_m1; Arntl Mm00500226_m1; Glpr2 Mm01329475_m1; Gja1 Mm01179639_s1; Ednrb Mm00432989; S100b Mm00485897_m1; Sox10 Mm00569909_m1. Real-time PCR analysis was performed using ABI Prism 7900HT Sequence Detection System or StepOne Real-Time PCR system (Applied Biosystems).

ILC3 Activation and Cell Signalling:

Sorted intestinal ILC3 cells were starved for 3 hours in RPMI at 37° C. in order to ensure ILC3 viability. Ret^{fl} or Ret^{Δ} were analysed directly ex vivo. To test ERK, AKT, p38-MAPK (Cell Signaling Technology) and STAT3 (BD Pharmigen) upon GFL stimulation WT ILC3 were activated with 500 ng/mL (each GFL) and co-receptors (rrGFR-α1, rmGFR-α2, rrGFR-α3 and rrGNDF from R&D Systems; rhNRTN and rhARTN from PeproTech) for 10 and 30 min. When referring to the use of ‘GFL’, we have employed GDNF, NRTN, ARTN and their specific co-receptors in combination. For inhibition experiments cells were incubated 1 h at 37° C. before GFL stimulation, to test ERK, AKT, p38/MAPK and STAT3 phosphorylation, or during overnight stimulation with GFLs, to determine Il22 expression levels. Inhibitors were purchased from Sigma-Aldrich: p38 MAPK/ERK-AKT-LY294002 (LY); ERK-PD98059 (PD); AKT-AKT Inhibitor VIII (VIII); p38 MAPK-SB 202190 (SB); and pSTAT3-S3I-201 (S3I).

Chromatin Immunoprecipitation (ChIP) Assay:

Enteric ILC3 from adult C57BL/6J mice were isolated by flow cytometry. Cells were starved for 3 h with RPMI supplemented with 1% hepes, sodium pyruvate, glutamine, streptomycin and penicillin and 0.1% 3-mercaptoethanol (Gibco) at 37° C. Cells were stimulated with GFLs (500 ng/mL each)⁸, lysed, cross-linked and chromosomal DNA-protein complex sonicated to generate DNA fragments ranging from 100-300 base pairs. DNA/protein complexes were immunoprecipitated, using LowCell# ChIP kit (Diagenode)¹⁸, with 3 μg of rabbit polyclonal antibody against anti-pSTAT3 (Cell Signalling Technology), rabbit control IgG (Abcam) or H3K36me3 (07-030; Millipore). Immunoprecipitates were uncross-linked and analysed by quantitative PCR using primer pairs (5′-3′) flanking putative sites on Il22. Vehicle (BSA) stimulated ILC3s were used as controls. Il22 primer sequences were previously described⁴⁸⁻⁵⁰, briefly:

a,
(SEQ ID NO: 1)
F-TGCAATCAATCCCAGTATTTTG
and
(SEQ ID NO: 2)
R-CTTGTGCAAGCATAAGTCTCAA;
b,
(SEQ ID NO: 3)
F-GAAGTTGGTGGGAAAATGAGTCCGTGA
and
(SEQ ID NO: 4)
R-GCCATGGCTTTGCCGTAGTAGATTCTG;
c,
(SEQ ID NO: 5)
F-ACGGGAGATCAAAGGCTGCTCT
and
(SEQ ID NO: 6)
R-GCCAACAAGGTGCTTTTGC;
d,
(SEQ ID NO: 7)
F-CTCACCGTGACGTTTTAGGG
and
(SEQ ID NO: 8)
R-GTGAATGATATGACATCAGAC;
e,
(SEQ ID NO: 9)
F-CGACGAACATGCTCCCCTGATGTTTTT
and
(SEQ ID NO: 10)
R-AAACTCATAGATTTCTGCAGGACAGCC;
f,
(SEQ ID NO: 11)
F-AGCTGCATCTCTTTCTCTCCA
and
(SEQ ID NO: 12)
R-TATCCTGAAGGCCAAAATAGGA;
g,
(SEQ ID NO: 13)
F-ACGACCAGAACATCCAGAAGA
and
(SEQ ID NO: 14)
R-GCAGAGAAAGAAATCCCCGC;
h,
(SEQ ID NO: 15)
F-AGGGGGACTTGCTTTGCCATTT
and
(SEQ ID NO: 16)
R-AACACCCCTTCTTTCCTCCTCCAT;
i,
(SEQ ID NO: 17)
F-CTGCTCCTTCCTGCCTTCTA
and
(SEQ ID NO: 18)
R-CTGAGCCAGGTTTCATGTGA.

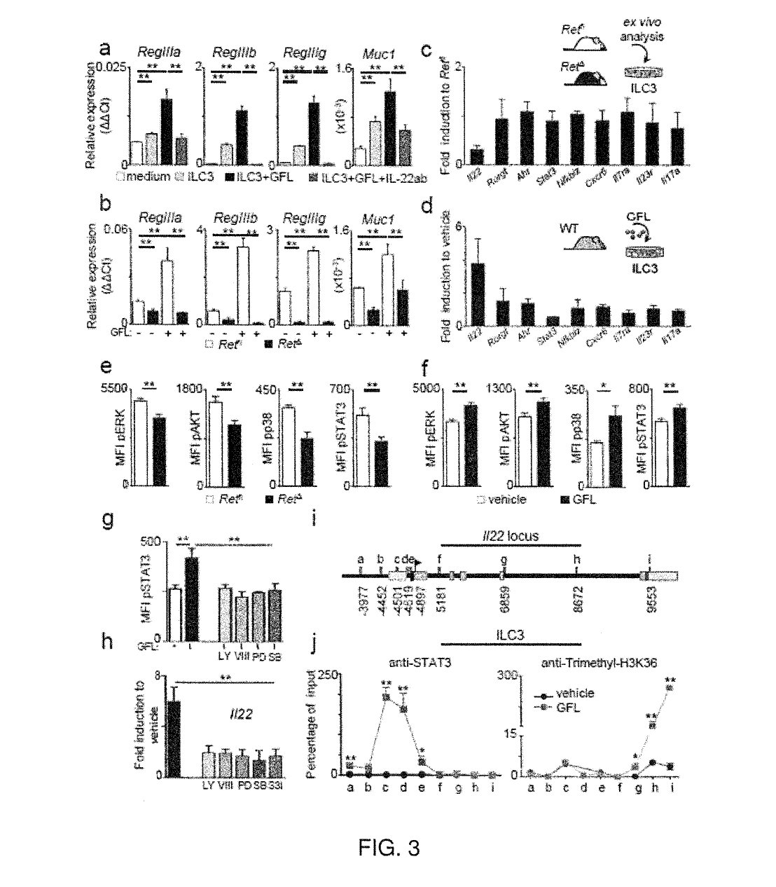

Primer positions are shown in FIG. 3i relative to the transcription start codon of Il22.

Colony Forming Units and Paracellular Permeability:

Organs were harvested, weighed, and brought into suspension. Bacterial colony forming units (CFU) were determined per gram of tissue and total organ. CFU were determined via serial dilutions on Luria Broth (LB) agar and MacConkey agar (Sigma-Aldrich). Colonies were counted after 2 days of culture at 37° C. To address intestinal paracellular permeability 16 mg per mouse of Dextran-Fitc (Sigma Aldrich) were administrated by gavage after overnight starvation. Plasma was analysed after 4 hours of Dextran-Fitc administration using a Microplate Reader TECAN Infinity F500.

BrdU Administration and Ki-67 Labeling:

BrdU was administrated by i.p. injection (1.25 mg/mouse). For flow cytometric analysis of epithelial cell proliferation anti-BrdU (Staining Kit for flow Cytometry—eBioscience) and anti-mouse Ki-67 antibody (BioLegend) were employed.

Quantitative PCR Analysis of Bacteria in Stool at the Phylum Level:

DNA from faecal pellet samples was isolated with ZR Fecal DNA MicroPrep™ (Zymo Research). Quantification of bacteria were determined from standard curves established by qPCR. qPCR were performed with Power SYBR® Green PCR Master Mix (Applied Biosystems) and different primer sets using a StepOne Plus (Applied Biosystems) thermocycler. Samples were normalized to 16S rDNA and reported according to the 2^{-ΔΔCT} method. Primer sequences were:

16S rDNA,
(SEQ ID NO: 19)
F-ACTCCTACGGGAGGCAGCAGT
and
(SEQ ID NO: 20)
R-ATTACCGCGGCTGCTGGC;
Firmicutes,
(SEQ ID NO: 21)
F-ACTCCTACGGGAGGCAGC
and
(SEQ ID NO: 22)
R-GCTTCTTAGTCAGGTACCGTCAT;
Bacteroidetes,
(SEQ ID NO: 23)
F-GGTTCTGAGAGGAGGTCCC
and
(SEQ ID NO: 24)
R-GCTGGCTCCCGTAGGAGT;
Proteobacteria,
(SEQ ID NO: 25)
F-GGTTCTGAGAGGAGGTCCC
and
(SEQ ID NO: 26)
R-GCTGGCTCCCGTAGGAGT.

16S rRNA Quantification and Gene Sequencing:

Faeces were isolated from co-housed Ret^{fl} or Ret^{Δ} littermates. Sequencing of the 16S rRNA gene was performed as previously described⁵¹. Briefly, barcoded primers were used to amplify the V4 region of the 16S rRNA gene, and the amplicons were sequenced on a MiSeq instrument (Illumina, San Diego, USA) using 150 bp, paired-end chemistry at the University of Pennsylvania Next Generation Sequencing Core. The paired ends were assembled and quality filtered, selecting for reads with a quality score ≥30. Reads with >10 bp homopolymers and reads shorter than 248 bp or longer than 255 bp were removed from the analysis. 16S rRNA sequence data were processed using mothur v 1.25.0⁵² and QIIME v 1.8⁵³. Chimeric sequences were removed with ChimeraSlayer⁵⁴. Operational taxonomic units (OTUs) were defined with CD-HIT⁵⁵ using 97% sequence similarity as a cut-off. Only OTUs containing ≥2 sequences were retained; OTUs assigned to Cyanobacteria or which were not assigned to any phylum were removed from the analysis. Taxonomy was assigned using the Ribosomal Database Project (RDP) classifier v 2.2⁵⁶, multiple sequence assignment was performed with PyNAST (v 1.2.2)⁵⁷, and FastTree⁵⁸ was used to build the phylogeny. Samples were rarified to 22,000 sequences per sample for alpha- and beta-diversity analyses. Taxonomic relative abundances are reported as the median with standard deviation. P values were calculated using the Wilcoxon rank-sum test. Statistical tests were conducted in R v. 3.2.0. To determine which factors were associated with microbial community composition, statistical tests were performed using the non-parametric analysis of similarities (ANOSIM) with weighted UniFrac distance metrics⁵⁹.

Data Accession:

The sequencing data generated in this study have been submitted to the NCBI Sequence Read Archive under BioProject PRJNA314493 (SRA: http://www.ncbi.nlm.nih.gov/sra/?term=PRJNA314493).

Intestinal Organoids:

IntestiCult™ Organoid Growth Medium and Gentle Cell Dissociation Reagent were purchased from StemCell. Intestinal crypts were isolated from C57BL/6J mice according to the manufacturer's instructions and were added to previously thawed, ice-cold Matrigel at a 1:1 ratio and at a final concentration of 5,000-7,000 crypts/mL. 15 μL of this mix was plated per well of a 96 well round-bottom plate. After Matrigel solidification 100 μL of growth medium (100 U/mL penicillin/streptomycin) was added and replaced every 3 days. Organoids were grown at 37° C. with 5% C02 and passaged according to the manufacturer's instructions. Freshly sorted intestinal ILC3 were added to 5-8 days old epithelial organoids after plating for 24 hours with or without anti-mouse IL-22 antibody (R&D Systems).

IL-22 Agonist Administration In Vivo:

150 μg of anti-IL-22 antibody (8E 11; gift from Genentech, South San Francisco, Calif.) or mouse IgG isotype control (MOPC-21; Bio X Cell) was administered i.p. to Ret^{MEN2B} mice every 2 days. Animals were analysed 2 weeks after the first administration.

Neurosphere-Derived Glial Cells:

Neurosphere-derived glial cells were obtained as previously described⁶⁰. Briefly, total intestines from E14.5 C57BL/6J and Myd88^{-/-} mice were digested with collagenase D (0.5 mg/mL; Roche) and DNase I (0.1 mg/mL; Roche) in DMEM/F-12, GlutaMAX, supplemented with 1% hepes, streptomycin/penicillin and 0.1% β-mercaptoethanol (Gibco) for approximately 30 minutes at 37° C. under gentle agitation. Cells were cultured during 1 week in a CO2 incubator at 37° C. in DMEM/F-12, GlutaMAX™, streptomycin and penicillin and 0.1% P3-mercaptoethanol (Gibco) supplemented with B27 (Gibco), EGF (Gibco) and FGF2 (Gibco) 20 ng/mL. After 1 week of culture cells were treated with 0.05% trypsin (Gibco), transferred into PDL (Sigma-Aldrich) coated plates and culture in DMEM supplemented with 10% FBS, 1% hepes, glutamine, streptomycin and penicillin and 0.1% β-mercaptoethanol (Gibco) until confluence. Glial cells were activated with TLR2 (5 μg/ml) (Pam3CSK4), TLR3 (100 g/ml) (PolyI:C), TLR4 (50 ng/ml) (LPS), TLR9 (50 g/ml) (DsDNA-EC) ligands from Invivogen and IL-1β (10 ng/mL) (401ML005), IL-18 (50 ng/mL) (B002-5), IL-33 (0.1 ng/mL) (3626ML) recombinant proteins from R&D Systems. Cells were also co-cultured with purified ILC3 from WT and Illb deficient mice. IL-22 expression in glial-ILC3 co-cultures upon TLR4 activation was also performed using GDNF (2 g/mL) (AB-212-NA), NRTN (2 g/mL) (AF-387sp) and ARTN (0.3 μg/mL) (AF-1085-sp) blocking antibodies. Cells were analysed after 24 hours of co-culture.

Statistics:

Results are shown as mean±SEM. Statistical analysis used Microsoft Excel. Variance was analysed using F-test. Student's t-test was performed on homocedastic populations, and Student's t-test with Welch correction was applied on samples with different variances. Analysis of survival curves was performed using a MAntel-Cox test. Results were considered significant at *p≤0.05; **p≤0.01. Statistical treatment of metagenomics analysis is described in the methods section: 16S rRNA gene sequencing and analysis.

### Example 1: The Neurotrophic Factor Receptor RET Drives Enteric ILC3-Derived IL-22

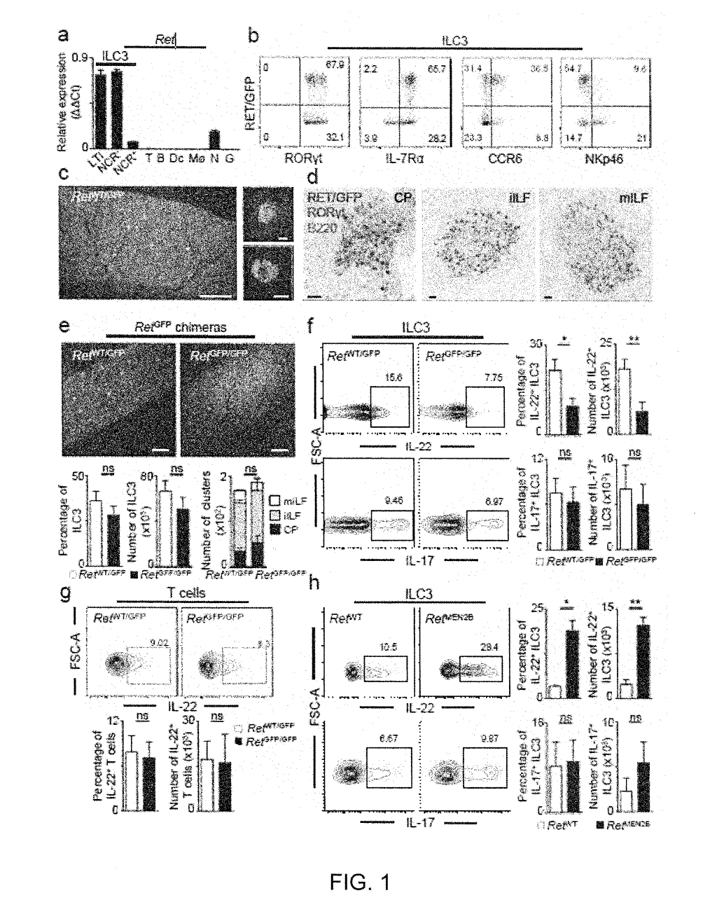

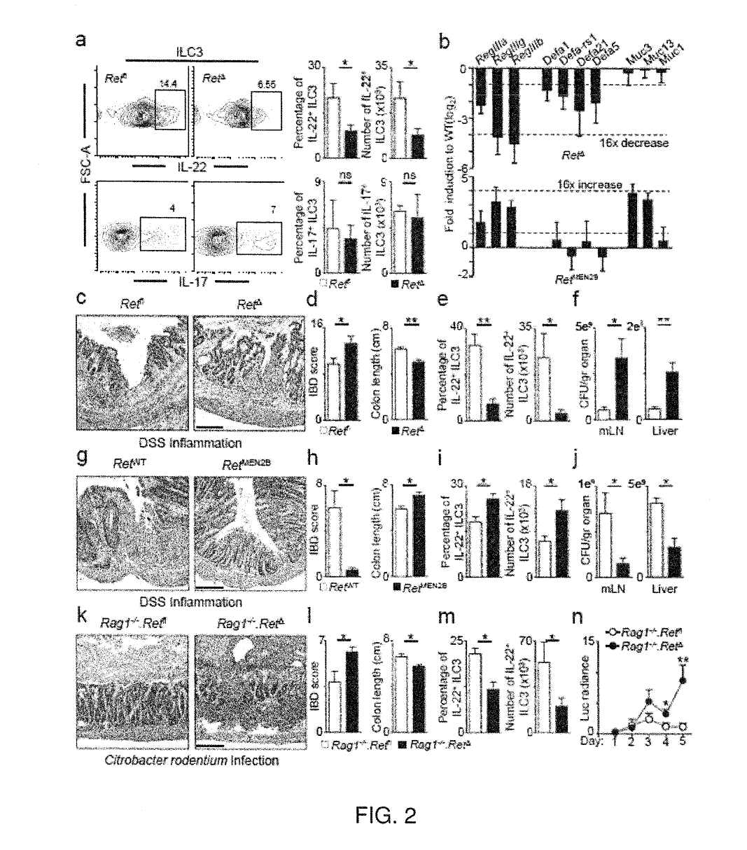

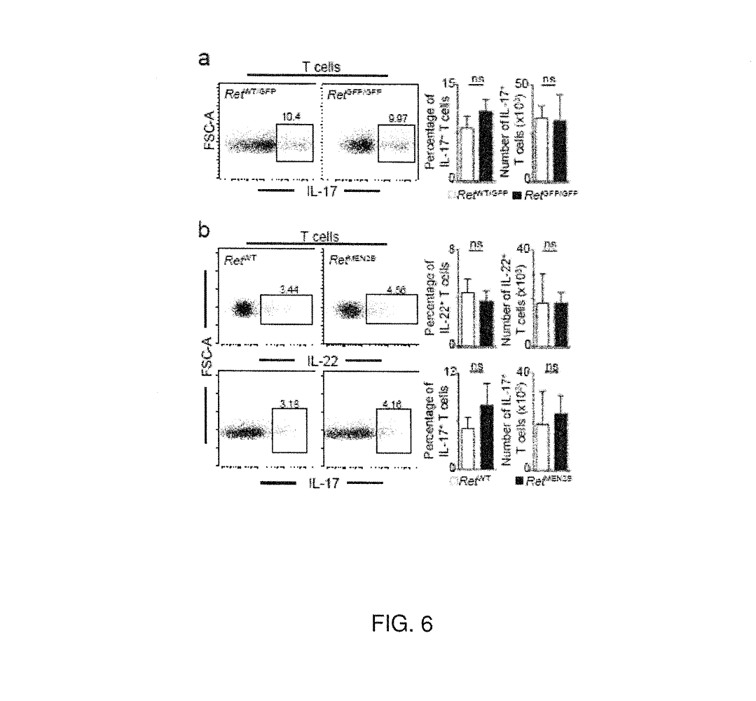

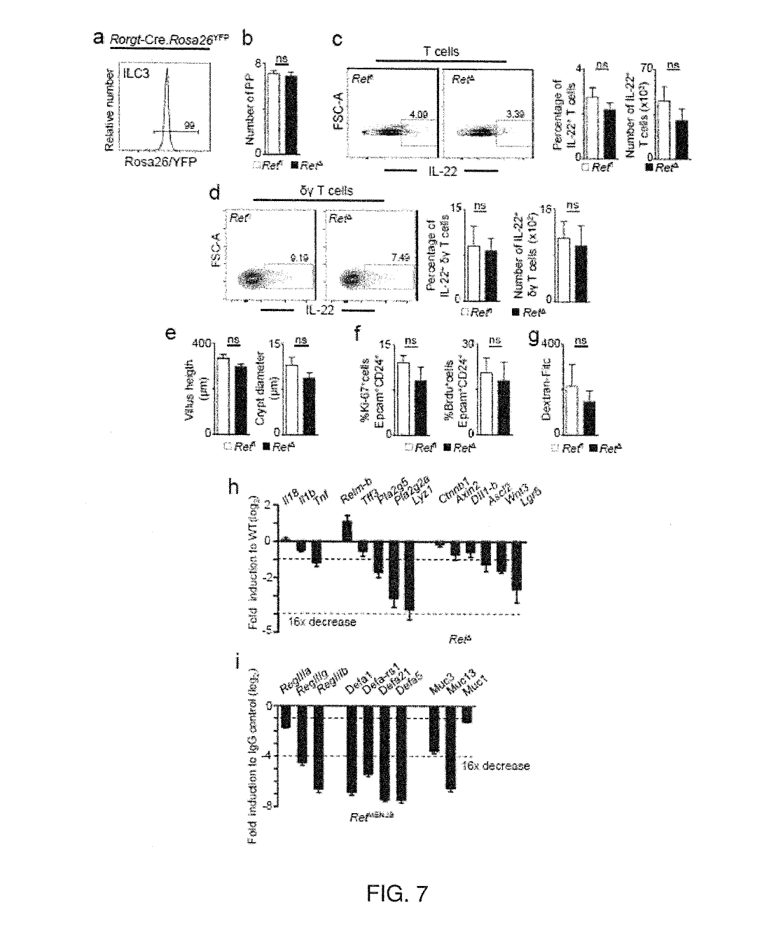

Analysis of gut lamina propria revealed that ILC3 express high levels of Ret (FIG. 1a)^{7,12}, a finding confirmed at the protein level and by Ret^{GFP} knock-in mice (FIGS. 1b-1d and FIG. 5a-5d)¹³. ILC3 subsets expressed Ret^{GFP} and aggregated in Cryptopatches (CP) and Isolated Lymphoid Follicles (ILF), suggesting a role of neuroregulators in ILC3 (FIGS. 1b-1d and FIGS. 5b-5j). To explore this hypothesis, foetal liver cells were transplanted from Ret competent (Ret^{WT/GFP}) or deficient (Ret^{GFP/GFP})¹³ animals into alymphoid Rag1^{-/-}γc^{-/-} hosts. Ret deficient chimeras revealed unperturbed ILC3 and CP development (FIG. 1e). Strikingly, IL-22 expressing ILC3 were largely reduced despite normal IL-22 producing T cells (FIGS. 1f,1g). In contrast, innate IL-17 was unaffected by Ret ablation (FIG. 1f and FIG. 6a). In agreement, analysis of gain-of-function Ret^{MEN2B} mice¹⁴ revealed a selective increase of IL-22 producing ILC3 while their IL-17 counterparts were unaffected (FIG. 1h and FIG. 6b). To more specifically evaluate the effects of RET in ILC3, Ret was deleted in RORγt expressing cells by breeding Rorgt-Cre to Ret^{fl/fl} mice^{15,16} (FIGS. 7a,7b). Analysis of Rorgt-Cre.Ret^{fl/fl} (Ret^{Δ}) mice revealed selective and large reduction of ILC3-derived IL-22, but normal IL-22 producing T cells (FIG. 2a and FIGS. 7c,7d). IL-22 acts on epithelial cells to induce reactivity and repair genes¹. When compared to their wild-type (WT) littermate controls, the Ret^{Δ} epithelium revealed normal morphology, proliferation and paracellular permeability, but a profound reduction of epithelial reactivity and repair genes (FIG. 2b and FIGS. 7e-7h). Accordantly, the Ret^{MEN2B} epithelium displayed increased levels of these molecules in an IL-22 dependent manner (FIG. 2b and FIG. 7i). These results indicate that RET signals selectively control innate IL-22 and shape intestinal epithelial reactivity.

### Example 2: ILC3-Intrinsic RET Signals Regulate Gut Defence and Homeostasis

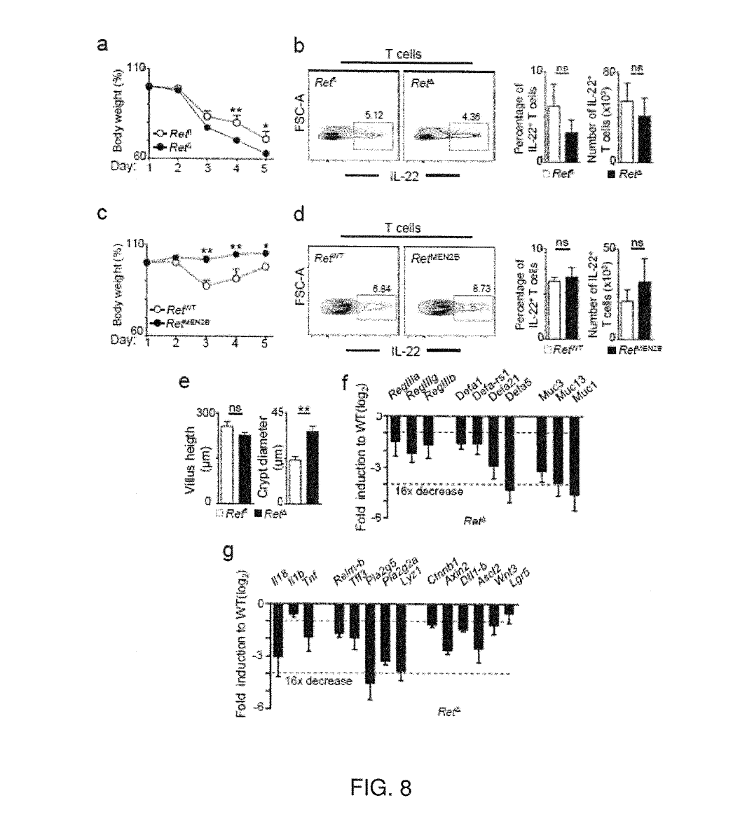

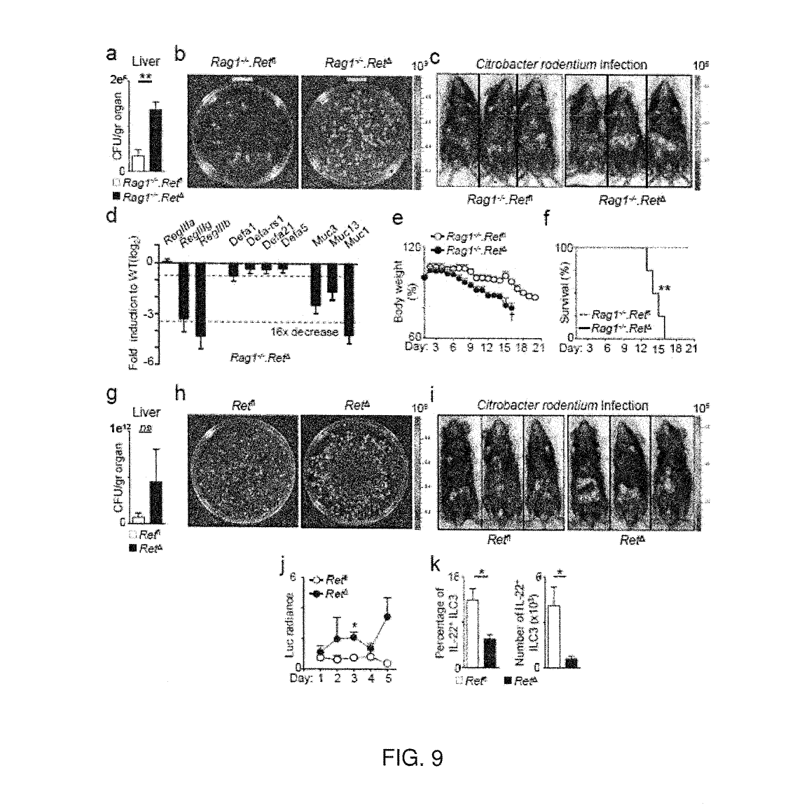

To interrogate whether neurotrophic factors regulate intestinal defence, how varying degrees of RET signals control enteric aggressions was tested. While Ret^{Δ} mice treated with Dextran Sodium Sulfate (DSS) had increased weight loss and inflammation, reduced IL-22 producing ILC3, decreased epithelial reactivity/repair genes and pronounced bacterial translocation from the gut, Ret^{MEN2B} mutants were highly protected over their WT littermate controls (FIGS. 2c-2j and FIG. 8). Since DSS mostly causes epithelial injury, whether ILC3-autonomous RET signals are required to control infection was tested. To this end, Ret^{Δ} mice were bred to Rag1^{-/-} mice to formally exclude adaptive T cell effects. Rag1^{-/-}.Ret^{Δ} mice were infected with the attaching and effacing bacteria Citrobacter rodentium. When compared to their littermate controls, Rag1^{-/-}.Ret^{Δ} mice had marked gut inflammation, reduced IL-22 producing ILC3, increased C. rodentium infection and translocation, reduced epithelial reactivity genes, increased weight loss and reduced survival (FIGS. 2k-2n and FIG. 9). Altogether, these data indicate that ILC3-intrinsic neurotrophic factor cues regulate gut defence and homeostasis.

### Example 3: RET Signals Control ILC3 Function and Gut Defence Via Direct Regulation of Il22

Formal definition that IL-22 is the molecular link between RET-dependent ILC3 activation and epithelial reactivity was provided by a multi-tissue organoid system. Addition of GFL to ILC3/epithelial organoids strongly induced epithelial reactivity genes in an IL-22 and RET dependent manner (FIGS. 3a,3b and FIG. 10a). To further examine how RET signals control innate IL-22 a gene signature associated with ILC identity¹ was investigated. While most of those genes were unperturbed, notably the master ILC transcription factors Runx1, Id2, Gata3, Rora, Rorgt, Ahr and Stat3, Il22 was significantly reduced in Ret^{Δ} ILC3 (FIG. 3c and FIG. 10b). In agreement, activation of ILC3 with all or distinct GFL/GFRα pairs in trans efficiently increased Il22 despite normal expression of other ILC3-related genes (FIG. 3d and FIG. 10c). Activation of RET by GFL leads to p38 MAPK/ERK-AKT cascade activation in neurons, while phosphorylation of STAT3 shapes Il22 expression^{7,17}. Analysis of Ret^{Δ} ILC3 revealed hypo-phosphorylated ERK1/2, AKT, p38/MAP kinase and STAT3 (FIG. 3e and FIG. 10d). Accordantly, GFL-induced RET activation in ILC3 led to rapid ERK1/2, AKT, p38/MAP kinase and STAT3 phosphorylation and increased Il22 transcription (FIGS. 3d,3f and FIGS. 10e,10f). In agreement, inhibition of ERK, AKT or p38/MAP kinase upon GFL activation led to impaired STAT3 activation and Il22 expression (FIGS. 3g,3h). Finally, inhibition of STAT3 upon GFL-induced RET activation led to decreased Il22 (FIG. 3h). To examine whether GFL directly regulate Il22 chromatin immunoprecipitation (ChIP) was performed (FIGS. 3i,3j)¹⁸. Stimulation of ILC3 with GFL resulted in increased binding of pSTAT3 in the Il22 promoter and increased trimethyl-H3K36 at the 3′ end of Il22, indicating active Il22 transcribed regions (FIGS. 3d,3j)¹⁹. Thus, cell-autonomous RET signals control ILC3 function and gut defence via direct regulation of Il22 downstream of STAT3 activation.

### Example 4: Mucosal Glial Cells Orchestrate Innate IL-22 Via Neurotrophic Factors

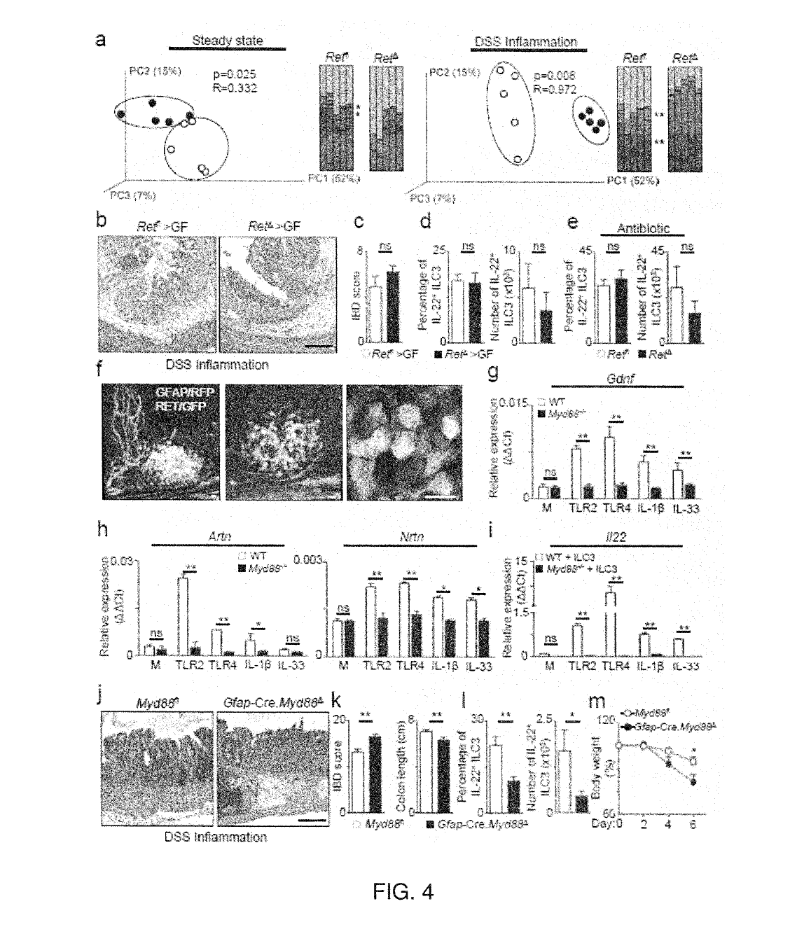

Propensity to inflammation and dysregulation of intestinal homeostasis have been associated to dysbiosis^{20,21}. When compared to their WT littermates, Ret^{Δ} mice have altered microbial communities as evidenced by quantitative analysis, weighted UniFrac analysis and significantly altered levels of Sutterella, unclassified Clostridiales and Bacteroides (FIG. 4a and FIG. 1l). Discrete microbial communities may have transmissible colitogenic potential^{20,21}. Nevertheless, germ-free mice colonised with the microbiota of Ret^{Δ} or their control littermates revealed similar susceptibility to DSS-induced colitis and identical innate IL-22 (FIGS. 4b-4d). In agreement, co-housed Ret^{Δ} and WT littermates had differential propensity to intestinal inflammation (FIGS. 2c,2d). Together, these data indicate that dysbiosis per se is insufficient to cause altered innate IL-22 and susceptibility to gut inflammation as observed in Ret^{Δ} mice (FIGS. 2c-2f). Thus, it was hypothesised that GFL producing cells integrate commensal and environmental signals to control innate IL-22. Accordingly, antibiotic treatment of Ret^{Δ} and their WT littermate controls resulted in similar ILC3-derived IL-22 (FIG. 4e)²².

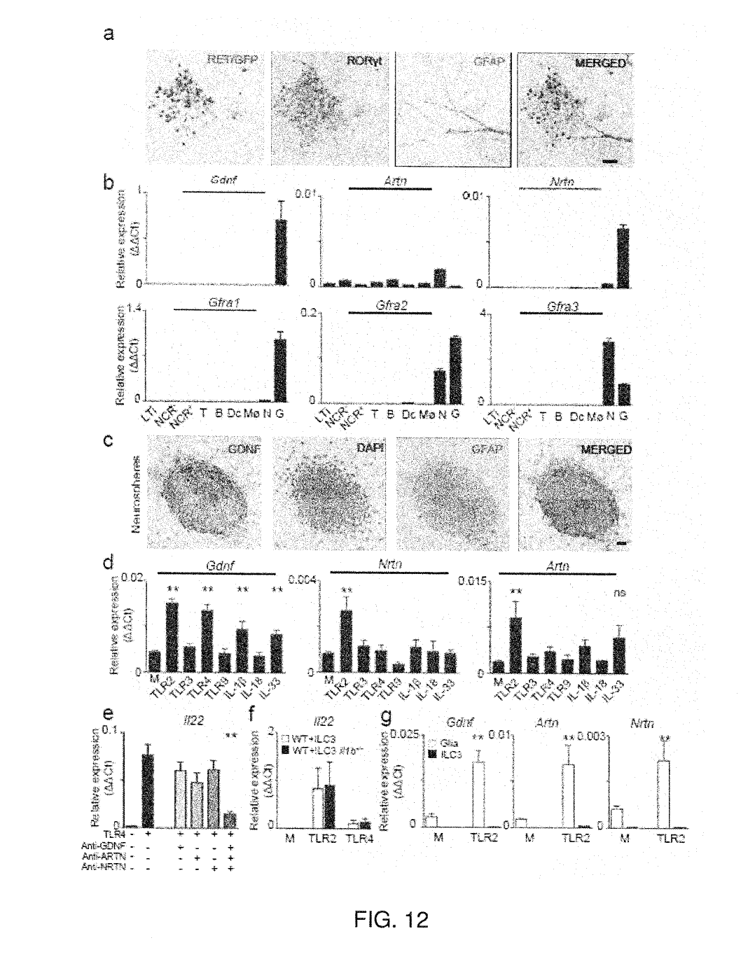

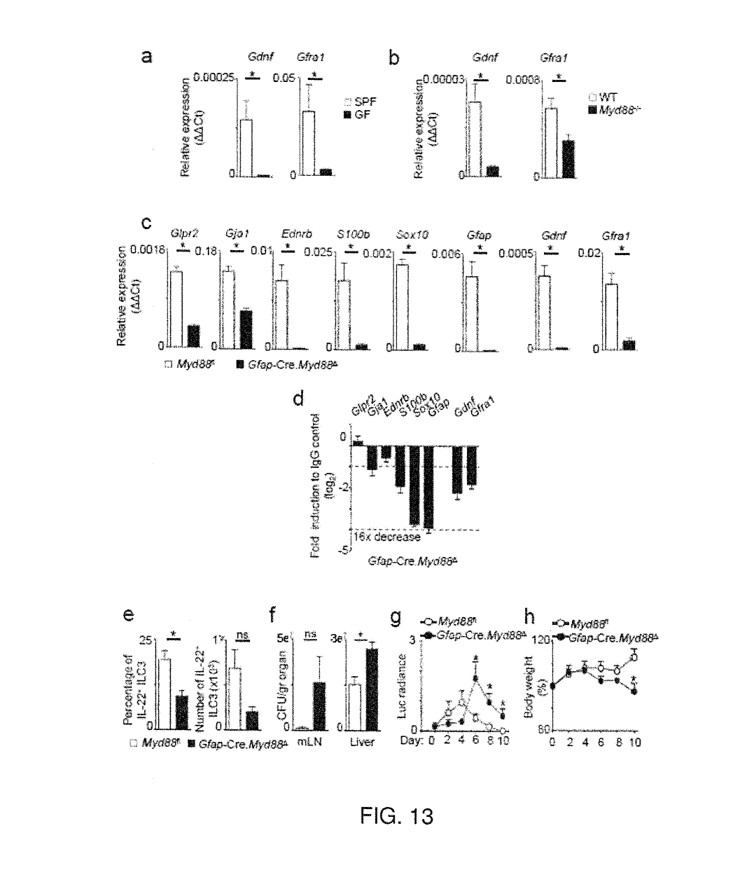

Neurotrophic factors of the GDNF family were shown to be produced by enteric glial cells, which are neuron-satellites expressing the glial fibrillary acidic protein (GFAP)^{7,23}. Strikingly, double reporter mice for ILC3 (Ret^{GFP}) and glial cells (Gfap-Cre.Rosa26^{RFP}) revealed that stellate-shaped projections of glial cells are adjacent (4.35 μm±+1.42) to RORγt⁺ILC3 within CP (FIG. 4f and FIG. 12a). These data suggest a paracrine glial-ILC3 crosstalk orchestrated by neurotrophic factors. In agreement, lamina propria glial cells were main producers of GFL (FIG. 12b). Recent studies have shown that glial cells express pattern recognition receptors, notably Toll-like receptors (TLRs)^{24,25}. Activation of neurosphere-derived glial cells revealed they specifically respond to TLR2, TLR4, and the alarmins IL-1β and IL-33, which efficiently controlled GFL expression and induced robust innate Il22 in a MYD88 dependent manner (FIGS. 4g-4i and FIGS. 12c-12g). To formally demonstrate the physiological importance of MYD88-dependent glial cell sensing on innate IL-22, Myd88 was deleted in GFAP expressing glial cells by breeding Gfap-Cre to Myd88^{fl/fl} mice^{26,27}. Remarkably, glial-intrinsic deletion of Myd88 resulted in decreased intestinal GFL, increased gut inflammation, impaired ILC3-derived IL-22, and increased weight loss (FIGS. 4j-4m; FIGS. 13a-13d). In agreement, Gfap-Cre.Myd88a mice had increased susceptible to C. rodentium infection (FIGS. 13e-13h). Thus, mucosal glial cells orchestrate innate IL-22 via neurotrophic factors, downstream of MYD88-dependent sensing of commensal products and alarmins.

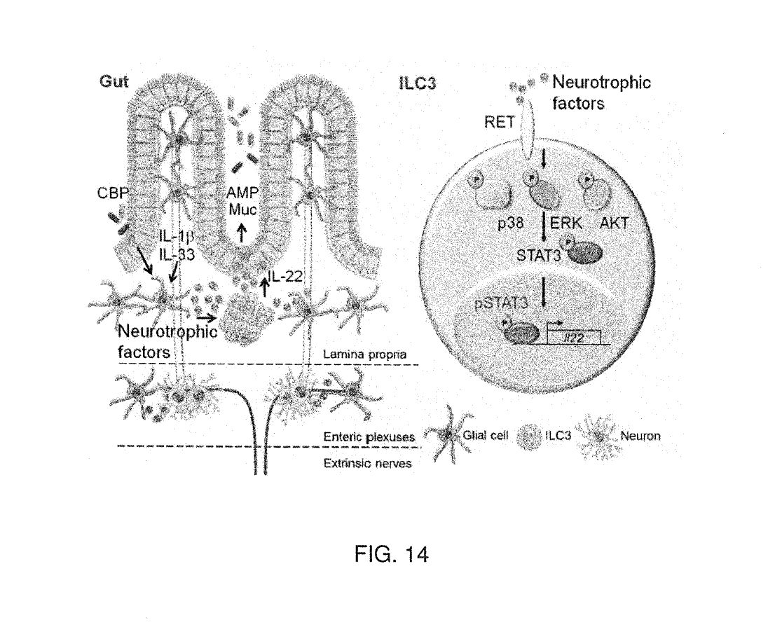

Defining the mechanisms by which ILC3 integrate environmental cues is critical to understand mucosal homeostasis. This work sheds light on the relationships between ILC3 and their microenvironment, notably through decoding a novel glial-ILC3-epithelial cell unit orchestrated by neurotrophic factors (FIG. 14). Glial-derived neurotrophic factors operate in an ILC3-intrinsic manner by activating the tyrosine kinase RET, which directly regulates innate IL-22 downstream of p38 MAPK/ERK-AKT and STAT3 phosphorylation (FIG. 14).

### REFERENCES

1 Artis, D. & Spits, H. The biology of innate lymphoid cells. Nature 517, 293-301 (2015).
2 van de Pavert, S. A. et al. Maternal retinoids control type 3 innate lymphoid cells and set the offspring immunity. Nature 508, 123-127 (2014).
3 Spencer, S. P. et al. Adaptation of innate lymphoid cells to a micronutrient deficiency promotes type 2 barrier immunity. Science 343, 432-437 (2014).
4 Kiss, E. A. et al. Natural aryl hydrocarbon receptor ligands control organogenesis of intestinal lymphoid follicles. Science 334, 1561-1565 (2011).
5 Lee, J. S. et al. AHR drives the development of gut ILC22 cells and postnatal lymphoid tissues via pathways dependent on and independent of Notch. Nat Immunol 13, 144-151 (2011).
6 Qiu, J. et al. The aryl hydrocarbon receptor regulates gut immunity through modulation of innate lymphoid cells. Immunity 36, 92-104 (2011).
7 Mulligan, L. M. RET revisited: expanding the oncogenic portfolio. Nat Rev Cancer 14, 173-186 (2014).
8 Fonseca-Pereira, D. et al. The neurotrophic factor receptor RET drives haematopoietic stem cell survival and function. Nature 514, 98-101 (2014).
9 Veiga-Fernandes, H. et al. Tyrosine kinase receptor RET is a key regulator of Peyer's Patch organogenesis. Nature 446, 547-551 (2007).
10 Patel, A. et al. Differential RET signaling pathways drive development of the enteric lymphoid and nervous systems. Sci Signal 5, ra55 (2012).
11 Almeida, A. R. et al. The neurotrophic factor receptor RET regulates IL-10 production by in vitro polarised T helper 2 cells. Eur J Immunol 44, 3605-3613 (2014).
12 Robinette, M. L. et al. Transcriptional programs define molecular characteristics of innate lymphoid cell classes and subsets. Nat Immunol 16, 306-317 (2015).
13 Hoshi, M., Batourina, E., Mendelsohn, C. & Jain, S. Novel mechanisms of early upper and lower urinary tract patterning regulated by RetY1015 docking tyrosine in mice. Development 139, 2405-2415 (2012).
14 Smith-Hicks, C. L., Sizer, K. C., Powers, J. F., Tischler, A. S. & Costantini, F. C-cell hyperplasia, pheochromocytoma and sympathoadrenal malformation in a mouse model of multiple endocrine neoplasia type 2B. Embo J 19, 612-622 (2000).
15 Sawa, S. et al. Lineage relationship analysis of RORgammat+ innate lymphoid cells. Science 330, 665-669 (2010).
16 Almeida, A. R. et al. RET/GFRalpha signals are dispensable for thymic T cell development in vivo. PLoS One 7, e52949 (2012).
17 Rutz, S., Wang, X. & Ouyang, W. The IL-20 subfamily of cytokines—from host defence to tissue homeostasis. Nat Rev Immunol 14, 783-795 (2014).
18 Xu, W. et al. NFIL3 orchestrates the emergence of common helper innate lymphoid cell precursors. Cell Rep 10, 2043-2054 (2015).
19 Wen, H. et al. ZMYND11 links histone H3.3K36me3 to transcription elongation and tumour suppression. Nature 508, 263-268 (2014).
20 Garrett, W. S. et al. Enterobacteriaceae act in concert with the gut microbiota to induce spontaneous and maternally transmitted colitis. Cell Host Microbe 8, 292-300 (2010).
21 Elinav, E. et al. NLRP6 inflammasome regulates colonic microbial ecology and risk for colitis. Cell 145, 745-757 (2011).
22 Rakoff-Nahoum, S., Paglino, J., Eslami-Varzaneh, F., Edberg, S. & Medzhitov, R. Recognition of commensal microflora by toll-like receptors is required for intestinal homeostasis. Cell 118, 229-241 (2004).
23 Neunlist, M. et al. The digestive neuronal-glial-epithelial unit: a new actor in gut health and disease. Nature reviews. Gastroenterology & hepatology 10, 90-100 (2013).
24 Brun, P. et al. Toll-like receptor 2 regulates intestinal inflammation by controlling integrity of the enteric nervous system. Gastroenterology 145, 1323-1333 (2013).
25 Kabouridis, P. S. et al. Microbiota controls the homeostasis of glial cells in the gut lamina propria. Neuron 85, 289-295 (2015).
26 Zhuo, L. et al. hGFAP-cre transgenic mice for manipulation of glial and neuronal function in vivo. Genesis 31, 85-94 (2001).
27 Hou, B., Reizis, B. & DeFranco, A. L. Toll-like receptors activate innate and adaptive immunity by using dendritic cell-intrinsic and -extrinsic mechanisms. Immunity 29, 272-282 (2008).
28 van de Pavert, S. A. et al. Chemokine CXCL13 is essential for lymph node initiation and is induced by retinoic acid and neuronal stimulation. Nat Immunol 10, 1193-1199 (2009).
29 Bush, T. G. et al. Fulminant jejuno-ileitis following ablation of enteric glia in adult transgenic mice. Cell 93, 189-201 (1998).
30 Veiga-Fernandes, H. & Mucida, D. Neuro-Immune Interactions at Barrier Surfaces. Cell 165, 801-811 (2016).
31 Cao, X. et al. Defective lymphoid development in mice lacking expression of the common cytokine receptor gamma chain. Immunity 2, 223-238 (1995).
32 Mombaerts, P. et al. RAG-1-deficient mice have no mature B and T lymphocytes. Cell 68, 869-877. (1992).
33 Srinivas, S. et al. Cre reporter strains produced by targeted insertion of EYFP and ECFP into the ROSA26 locus. BMC Dev Biol 1, 4 (2001).
34 Madisen, L. et al. A robust and high-throughput Cre reporting and characterization system for the whole mouse brain. Nature neuroscience 13, 133-140 (2010).
35 Horai, R. et al. Production of mice deficient in genes for interleukin (IL)-1alpha, IL-1beta, IL-1alpha/beta, and IL-1 receptor antagonist shows that IL-1beta is crucial in turpentine-induced fever development and glucocorticoid secretion. The Journal of experimental medicine 187, 1463-1475 (1998).
36 Adachi, O. et al. Targeted disruption of the MyD88 gene results in loss of IL-1- and IL-18-mediated function. Immunity 9, 143-150 (1998).
37 Wiles, S., Pickard, K. M., Peng, K., MacDonald, T. T. & Frankel, G. In vivo bioluminescence imaging of the murine pathogen Citrobacter rodentium. Infect Immun 74, 5391-5396 (2006).
38 Collins, J. W. et al. Citrobacter rodentium: infection, inflammation and the microbiota. Nat Rev Microbiol 12, 612-623 (2014).
39 Ibiza, S. et al. Endothelial nitric oxide synthase regulates T cell receptor signaling at the immunological synapse. Immunity 24, 753-765 (2006).
40 Moolenbeek, C. & Ruitenberg, E. J. The Swiss Roll—a Simple Technique for Histological Studies of the Rodent Intestine. Lab Anim 15, 57-59 (1981).
41 Burich, A. et al. Helicobacter-induced inflammatory bowel disease in IL-10- and T cell-deficient mice. Am J Physiol Gastrointest Liver Physiol 281, G764-778 (2001).
42 Fort, M. M. et al. A synthetic TLR4 antagonist has anti-inflammatory effects in two murine models of inflammatory bowel disease. J Immunol 174, 6416-6423 (2005).
43 Seamons, A., Treuting, P. M., Brabb, T. & Maggio-Price, L. Characterization of dextran sodium sulfate-induced inflammation and colonic tumorigenesis in Smad3(−/−) mice with dysregulated TGFbeta. PLoS One 8, e79182 (2013).
44 Bogunovic, M. et al. Origin of the lamina propria dendritic cell network. Immunity 31, 513-525 (2009).
45 Muller, P. A. et al. Crosstalk between muscularis macrophages and enteric neurons regulates gastrointestinal motility. Cell 158, 300-313 (2014).
46 Sanos, S. L. & Diefenbach, A. Isolation of NK cells and NK-like cells from the intestinal lamina propria. Methods Mol Biol 612, 505-517 (2010).
47 Joseph, N. M. et al. Enteric glia are multipotent in culture but primarily form glia in the adult rodent gut. The Journal of clinical investigation 121, 3398-3411 (2011).
48 Escobar, T. M. et al. miR-155 activates cytokine gene expression in Th17 cells by regulating the DNA-binding protein Jarid2 to relieve polycomb-mediated repression. Immunity 40, 865-879 (2014).
49 Guo, X. et al. Induction of innate lymphoid cell-derived interleukin-22 by the transcription factor STAT3 mediates protection against intestinal infection. Immunity 40, 25-39 (2014).
50 Yeste, A. et al. IL-21 induces IL-22 production in CD4+ T cells. Nat Commun 5, 3753 (2014).
51 Misic, A. M. et al. The shared microbiota of humans and companion animals as evaluated from Staphylococcus carriage sites. Microbiome 3, 2 (2015).
52 Schloss, P. D. et al. Introducing mothur: open-source, platform-independent, community-supported software for describing and comparing microbial communities. Appl Environ Microbiol 75, 7537-7541 (2009).
53 Caporaso, J. G. et al. QIIME allows analysis of high-throughput community sequencing data. Nature methods 7, 335-336 (2010).
54 Haas, B. J. et al. Chimeric 16S rRNA sequence formation and detection in Sanger and 454-pyrosequenced PCR amplicons. Genome Res 21, 494-504 (2011).
55 Fu, L., Niu, B., Zhu, Z., Wu, S. & Li, W. CD-HIT: accelerated for clustering the next-generation sequencing data. Bioinformatics 28, 3150-3152 (2012).
56 Wang, Q., Garrity, G. M., Tiedje, J. M. & Cole, J. R. Naive Bayesian classifier for rapid assignment of rRNA sequences into the new bacterial taxonomy. Appl Environ Microbiol 73, 5261-5267 (2007).
57 Caporaso, J. G. et al. PyNAST: a flexible tool for aligning sequences to a template alignment. Bioinformatics 26, 266-267 (2010).
58 Price, M. N., Dehal, P. S. & Arkin, A. P. FastTree: computing large minimum evolution trees with profiles instead of a distance matrix. Mol Biol Evol 26, 1641-1650 (2009).
59 Lozupone, C., Hamady, M. & Knight, R. UniFrac—an online tool for comparing microbial community diversity in a phylogenetic context. BMC Bioinformatics 7, 371 (2006).
60 Mich, J. K. et al. Prospective identification of functionally distinct stem cells and neurosphere-initiating cells in adult mouse forebrain. eLife 3, e02669 (2014).

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method for increasing production of interleukin-22 (IL-22) by Group 3 innate lymphoid cells (ILC3s), comprising
contacting ILC3s with an agonist of rearranged during transfection (RET) in an amount effective to increase production of IL-22 by the ILC3s.

2. The method of claim 1, wherein the agonist of RET comprises
(1) a combination of a soluble GDNF Family binding Receptor alpha (GFRα) and a GFRα ligand (GFL) or an analog or mimetic thereof; or
(2) an antibody that specifically binds to RET and increases RET tyrosine kinase activity or an antigen-binding fragment thereof.

3. The method of claim 2, wherein the combination of a soluble GDNF Family binding Receptor alpha (GFRα) and a GFRα ligand or an analog or mimetic thereof comprises:
(1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or
(2) a combination of two or more of (a), (b), (c), (d), (e), (f) and (g).

4. The method of any one of claims 1-3, wherein the contacting is in vitro.

5. The method of any one of claims 1-3, wherein the contacting is in vivo.

6. The method of claim 5, wherein the agonist is administered to a subject.

7. The method of claim 6, wherein the subject is a human.

8. The method of claim 6 or claim 7, wherein the subject is not otherwise in need of treatment with the agonist.

9. A method for treating a disease associated with Group 3 innate lymphoid cells (ILC3s), comprising
administering to a subject in need of such treatment an agonist of rearranged during transfection (RET) in an amount effective to treat the disease.

10. The method of claim 9, wherein the agonist of RET comprises
(1) a combination of a soluble GDNF Family binding Receptor alpha (GFRα) and a GFRα ligand or an analog or mimetic thereof; or
(2) an antibody that specifically binds to RET and increases RET tyrosine kinase activity or an antigen-binding fragment thereof.

11. The method of claim 10, wherein the combination of a soluble GDNF Family binding Receptor alpha (GFRα) and a GFRα ligand or an analog or mimetic thereof comprises:
(1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or
(2) a combination of two or more of (a), (b), (c), (d), (e), (f) and (g).

12. The method of any one of claims 9-11, wherein the subject is a human.

13. The method of any one of claims 9-12, wherein the disease is infection, inflammation, neoplasia, or altered gut physiology.

14. The method of any one of claims 9-13, wherein the subject is not otherwise in need of treatment with the agonist of RET.

15. The method of any one of claims 9-14, wherein the agonist of RET is administered intravenously, orally, nasally, rectally or through skin absorption.

16. An agonist of rearranged during transfection (RET) for use in treating a disease associated with Group 3 innate lymphoid cells (ILC3s), comprising administering to a subject in need of such treatment the agonist of RET in an amount effective to treat the disease.

17. The agonist of claim 16, wherein the agonist of RET comprises
(1) a combination of a soluble GDNF Family binding Receptor alpha (GFRα) and a GFRα ligand or an analog or mimetic thereof; or
(2) an antibody that specifically binds to RET and increases RET tyrosine kinase activity or an antigen-binding fragment thereof.

18. The agonist of claim 17, wherein the combination of a soluble GDNF Family binding Receptor alpha (GFRα) and a GFRα ligand or an analog or mimetic thereof comprises:
(1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or
(2) a combination of two or more of (a), (b), (c), (d), (e), (f) and (g).

19. The agonist of any one of claims 16-18, wherein the subject is a human.

20. The agonist of any one of claims 16-19, wherein the disease is infection, inflammation, neoplasia, or altered gut physiology.

21. The agonist of any one of claims 16-20, wherein the subject is not otherwise in need of treatment with the agonist of RET.

22. The agonist of any one of claims 16-21, wherein the agonist of RET is administered intravenously, orally, nasally, rectally or through skin absorption.

23. A method for treating a disease associated with Group 3 innate lymphoid cells (ILC3s), comprising
administering to a subject in need of such treatment a composition comprising ILC3s in an amount effective to treat the disease.

24. The method of claim 23, wherein the composition further comprises an agonist of rearranged during transfection (RET).

25. The method of claim 24, wherein the agonist of RET comprises
(1) a combination of a soluble GDNF Family binding Receptor alpha (GFRα) and a GFRα ligand or an analog or mimetic thereof; or
(2) an antibody that specifically binds to RET and increases RET tyrosine kinase activity or an antigen-binding fragment thereof.

26. The method of claim 25, wherein the combination of a soluble GDNF Family binding Receptor alpha (GFRα) and a GFRα ligand or an analog or mimetic thereof comprises:
(1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or
(2) a combination of two or more of (a), (b), (c), (d), (e), (f) and (g).

27. The method of any one of claims 23-26, wherein the subject is a human.

28. The method of any one of claims 23-27, wherein the disease is infection, inflammation, neoplasia, or altered gut physiology.

29. The method of any one of claims 23-28, wherein the subject is not otherwise in need of treatment with the ILC3s or the agonist of RET.

30. The method of any one of claims 23-29, wherein the ILC3s or the agonist of RET is administered intravenously, orally, nasally, rectally or through skin absorption.

31. A composition comprising activated Group 3 innate lymphoid cells (ILC3s) for use in treating a disease associated with ILC3s comprising administering to a subject in need of such treatment the composition comprising ILC3s in an amount effective to treat the disease.

32. The composition of claim 31, wherein the composition further comprises an agonist of rearranged during transfection (RET).

33. The method of claim 32, wherein the agonist of RET comprises
(1) a combination of a soluble GDNF Family binding Receptor alpha (GFRα) and a GFRα ligand or an analog or mimetic thereof; or
(2) an antibody that specifically binds to RET and increases RET tyrosine kinase activity or an antigen-binding fragment thereof.

34. The method of claim 33, wherein the combination of a soluble GDNF Family binding Receptor alpha (GFRα) and a GFRα ligand or an analog or mimetic thereof comprises:
(1) a combination of: (a) soluble GDNF Family binding Receptor alpha 1 (GFRα1) and glial cell line-derived neurotrophic factor (GDNF) or an analog or mimetic thereof; (b) soluble GFRα2 and neurturin (NTRN) or an analog or mimetic thereof; (c) soluble GFRα3 and artemin (ARTN) or an analog or mimetic thereof; (d) soluble GFRα4 and persephin (PSPN) or an analog or mimetic thereof; (e) a soluble GFRα and N(4)-(7-chloro-2-[(E)-2-(2-chloro-phenyl)-vinyl]-quinolin-4-yl)-N(1),N(1)-diethyl-pentane-1,4-diamine (XIB4035); (f) a soluble GFRα and a BT compound; (g) a soluble GFRα and an antibody that specifically binds to and dimerizes the GFRα; or
(2) a combination of two or more of (a), (b), (c), (d), (e), (f) and (g).

35. The composition of any one of claims 31-34, wherein the subject is a human.

36. The composition of any one of claims 31-35, wherein the disease is infection, inflammation, neoplasia, or altered gut physiology.

37. The composition of any one of claims 31-36, wherein the subject is not otherwise in need of treatment with the ILC3s or the agonist of RET.

38. The composition of any one of claims 31-37, wherein the ILC3s or the ILC3s and the agonist of RET is administered intravenously, orally, nasally, rectally or through skin absorption.

39. A method for decreasing production of interleukin-22 (IL-22) by Group 3 innate lymphoid cells (ILC3s), comprising
contacting ILC3s with an antagonist of rearranged during transfection (RET) in an amount effective to decrease production of IL-22 by the ILC3s.

40. The method of claim 39, wherein the antagonist of RET is (1) an antibody that specifically binds and inhibits: (a) RET tyrosine kinase activity, (b) a GDNF Family binding Receptor alpha (GFRα), or (c) a GFRα ligand, or an antigen-binding fragment thereof; (2) an inhibitory nucleic acid molecule that reduces expression, transcription or translation of RET, a GFRα, or a GFRα ligand; or (3) a RET tyrosine kinase inhibitor, optionally AST 487, motesanib, cabozantinib, vandetanib, ponatinib, sunitinib, sorafenib, or alectinib.

41. The method of claim 40, wherein the GFRα is GFRα1, GFRα2, GFRα3, or GFRα4; or wherein the GFRα ligand is glial cell line-derived neurotrophic factor (GDNF), neurturin (NTRN), artemin (ARTN), or persephin (PSPN).

42. The method of claim 40, wherein the inhibitory nucleic acid molecule is a sRNA, shRNA, or antisense nucleic acid molecule.

43. The method of any one of claims 39-42, wherein the contacting is in vitro.

44. The method of any one of claims 39-42, wherein the contacting is in vivo.

45. The method of claim 44, wherein the antagonist of RET is administered to a subject.

46. The method of claim 45, wherein the subject is a human.

47. The method of claim 45 or claim 46, wherein the subject is not otherwise in need of treatment with the antagonist of RET.

48. A method for treating a disease associated with Group 3 innate lymphoid cells (ILC3s), comprising
administering to a subject in need of such treatment an antagonist of rearranged during transfection (RET) in an amount effective to treat the disease.

49. The method of claim 48, wherein the antagonist of RET is
(1) an antibody that specifically binds and inhibits: (a) RET tyrosine kinase activity, (b) a GDNF Family binding Receptor alpha (GFRα), or (c) a GFRα ligand, or an antigen-binding fragment thereof;
(2) an inhibitory nucleic acid molecule that reduces expression, transcription or translation of RET, a GFRα, or a GFRα ligand; or
(3) a RET tyrosine kinase inhibitor, optionally AST 487, motesanib, cabozantinib, vandetanib, ponatinib, sunitinib, sorafenib, or alectinib.

50. The method of claim 49, wherein the GFRα is GFRα1, GFRα2, GFRα3, or GFRα4; or wherein the GFRα ligand is glial cell line-derived neurotrophic factor (GDNF), neurturin (NTRN), artemin (ARTN), or persephin (PSPN).

51. The method of claim 49, wherein the inhibitory nucleic acid molecule is a sRNA, shRNA, or antisense nucleic acid molecule.

52. The method of any one of claims 48-51, wherein the subject is a human.

53. The method of any one of claims 48-52, wherein the subject is not otherwise in need of treatment with the antagonist of RET.

54. The method of any one of claims 48-53, wherein the disease is epithelial intestinal cancer.

55. The method of any one of claims 48-54, wherein the antagonist of RET is administered intravenously, orally, nasally, rectally or through skin absorption.

56. An antagonist of rearranged during transfection (RET) for use in treating a disease associated with Group 3 innate lymphoid cells (ILC3) comprising administering to a subject in need of such treatment the antagonist of RET in an amount effective to treat the disease.

57. The method of claim 56, wherein the antagonist of RET is
(1) an antibody that specifically binds and inhibits: (a) RET tyrosine kinase activity, (b) a GDNF Family binding Receptor alpha (GFRα), or (c) a GFRα ligand, or an antigen-binding fragment thereof;
(2) an inhibitory nucleic acid molecule that reduces expression, transcription or translation of RET, a GFRα, or a GFRα ligand; or
(3) a RET tyrosine kinase inhibitor, optionally AST 487, motesanib, cabozantinib, vandetanib, ponatinib, sunitinib, sorafenib, or alectinib.

58. The method of claim 57, wherein the GFRα is GFRα1, GFRα2, GFRα3, or GFRα4; or wherein the GFRα ligand is glial cell line-derived neurotrophic factor (GDNF), neurturin (NTRN), artemin (ARTN), or persephin (PSPN).

59. The method of claim 57, wherein the inhibitory nucleic acid molecule is a sRNA, shRNA, or antisense nucleic acid molecule.

60. The method of any one of claims 56-59, wherein the subject is a human.

61. The method of any one of claims 56-60, wherein the subject is not otherwise in need of treatment with the antagonist of RET.

62. The method of any one of claims 56-61, wherein the disease is epithelial intestinal cancer.

63. The method of any one of claims 56-62, wherein the antagonist of RET is administered intravenously, orally, nasally, rectally or through skin absorption.
